(19) United States
(12) Patent Application Publication
(10) Pub. No.: US 2019/0231906 A1
(43) Pub. Date: Aug. 1, 2019

(54) NANOPARTICLE DIAGNOSTIC AND METHODS FOR TREATING DISEASE

(71) Applicant: TARVEDA THERAPEUTICS, INC., Watertown, MA (US)

(72) Inventors: Sudhakar Kadiyala, Newton, MA (US); Patrick Lim Soo, Ridgewood, NJ (US); Mark Iwicki, Wellesley, MA (US); Craig A. Dunbar, Needham, MA (US); Mark T. Bilodeau, Waltham, MA (US); Rajesh R. Shinde, Lexington, MA (US); Rossitza G. Alargova, Brighton, MA (US); Michelle Dupont, Salem, MA (US)

(21) Appl. No.: 16/381,332

(22) Filed: Apr. 11, 2019

Related U.S. Application Data
(63) Continuation of application No. 14/908,661, filed on Jan. 29, 2016.
(60) Provisional application No. 61/859,826, filed on Jul. 30, 2013.

(51) Int. Cl. A61K 49/06; A61K 49/00; A61K 31/555; A61K 9/16

(52) U.S. Cl. CPC A61K 49/06; A61K 49/0004; A61K 9/1641; A61K 49/0032; A61K 49/0054; A61K 31/555

(57) ABSTRACT

The present invention relates to the use of in vivo contrast agents in medical imaging in order to diagnose and treat disease, and to monitor and assess disease progression following treatment with a nanoparticle therapeutic agent comprising an active pharmaceutical agent. The present invention also relates to modulating nanoparticle tumor concentration by modulating the PEG density of the nanoparticles.

### CROSS-REFERENCE TO RELATED APPLICATIONS

This application is a continuation application of Ser. No. 14/908,661, filed Jan. 29, 2016, which is a national phase entry of PCT/US2014/048820, filed Jul. 30, 2014, which claims priority to U.S. 61/859,826, filed on Jul. 30, 2013, the contents of each of which are incorporated herein by reference in their entirety.

### FIELD OF THE INVENTION

The present disclosure relates to the use of in vivo contrast agents in medical imaging in order to assess disease states and provide tailored treatment therefor with a nanoparticle therapeutic agent comprising an active agent, such as a chemotherapeutic or radiotherapeutic agent. The active agent is released from the nanoparticles at target cells in a controlled fashion.

### BACKGROUND OF THE INVENTION

Nanoparticulate drug delivery systems are attractive in systemic drug delivery because of their ability to prolong drug circulation half-life, reduce non-specific uptake, and better localization at tumor sites for example, perhaps through an enhanced permeability and retention (EPR) effect. Nanoparticle delivery of diagnostic and therapeutic agents has also been shown to have lower toxicity when compared to delivery of their “naked” small molecule counterparts. The lower toxicity is attributed to the improved biodistribution and longer circulation half-life. However, there is relatively little information about the biodistribution of nanoparticles in patients. As more nanoparticle platforms are being developed for biomedical applications (e.g., cancer treatment), there is increasing interest in developing strategies to monitor and assess the efficacy of such nanoparticle therapeutic agents (NTA).

The evaluation of the stage of the disease and the assessment of treatment are major factors in diagnosing and treating the disease progression in patients. While the use of contrast agents enhance the sensitivity of the detection of the body tissue or organs using a diagnostic device, targeted drug delivery plays a major role in enhancing the drug targeting at the cell-specific level. There remains a need for non-invasive methods to predict the accumulation of NTA at tumor sites and thereby predicting the effectiveness of NTA.

### SUMMARY OF THE INVENTION

The present invention relates to a method for using a contrast agent, such as ferumoxytol or other imaging agent, to establish if a patient achieves sufficient accumulation of a drug delivery vehicle (e.g., via EPR) for the subsequent administration of a nanoparticle therapeutic agent (NTA). The present invention also relates to the in vivo diagnosis, assessment and/or monitoring of disease progression either before or following treatment with an NTA. The present invention also provides a method of modulating the accumulation of NTA at tumor sites.

In one aspect of the invention, a method of increasing the accumulation of a nanoparticle at a tumor site is provided. The method comprises administering a nanoparticle to the tumor site, wherein the nanoparticle comprises at least one PEG moiety and a PEG density of at least about 0.2 g/g/nm²or 0.2 units/nm². In some embodiments, the PEG density of the nanoparticle is increased to increase the accumulation of the nanoparticle at a tumor site. In some embodiments, the nanoparticle is a nanoparticle therapeutic agent (NTA) comprising at least one pharmaceutically active agent. In some embodiments, the PEG density of the nanoparticle is at least about 0.3 g/nm², 0.4 g/nm², or 0.5 g/nm², or at least about 0.3 units/nm², 0.4 units/nm², or 0.5 units/nm². In some embodiments, the tumor is a highly vascularized tumor. In some embodiments, the tumor is pancreatic, brain, breast, cervical, colon, esophageal, gallbladder, head and neck, kidney, liver, multiple myeloma, ovarian, prostate, thyroid or lung cancer.

In another aspect of the invention, provided is a method of selecting a subject to be treated with NTA, the method comprising:
(a) administering a contrast agent to the subject;
(b) measuring the level of accumulation of the contrast agent at at least one intended site of treatment with an imaging technique;
(c) selecting the subject for NTA treatment based on the level of the accumulation;
   wherein the intended site of treatment is a tumor site.

In some embodiments, the contrast agent and the NTA differ from one another based on at least one parameter by at least 2 folds. In some embodiments, the parameters are size, density, or surface charge.

In some embodiments, the method of selecting subjects to be treated with NTA further comprising measuring the level of accumulation of the contrast agent at a reference site. In some embodiments, the reference site is plasma, bone, or muscle. A subject whose level of accumulation of the contrast agent at the tumor site is higher than the level of accumulation of the contrast agent at the reference site is treated with NTA.

In some embodiments, the contrast agent comprises a moiety selected from a group consisting of a fluorescent, luminescent, radioactive, and magnetic moiety.

In some embodiments, the imaging technique selected from ultrasound, X-ray, single-photon emission tomography/computed tomography (SPECT/CT), positron emission tomography/computed tomography (PET/CT), positron emission tomography (PET), magnetic resonance imaging (MRI), computed tomography (CT), single-photon emission tomography (SPECT), fluorescence tomography, and fluorescence spectroscopy.

In some embodiments, the tumor is pancreatic cancer, lung cancer, brain cancer, breast cancer, cervical cancer, colon cancer, esophageal cancer, gallbladder cancer, head and neck cancer, kidney cancer, liver cancer, multiple myeloma, thyroid cancer, or ovarian cancer.

In some embodiments, the NTA cannot be detected with the imaging technique. in some embodiments, the NTA does not comprise any fluorescent, luminescent, radioactive, or magnetic moiety. In some embodiments, the NTA comprises a triple-targeted conjugate having the formula:
(X—Y—Z)
wherein:
X is a targeting ligand;
Y is a linker; and
Z is a pharmaceutically active agent.

In another aspect of the invention, provided is a method of treating cancer comprising:
(a) administering a contrast agent to a subject;
(b) measuring the level of accumulation of the contrast agent at each of the intended site of treatment; wherein the intended site of treatment is a tumor site.
(c) determine if the subject is suitable for NTA treatment on the basis of the level of accumulation measured in step (b);
(d) administering NTA to the subject if the subject is determined to be suitable for NTA treatment in step (c).

In some embodiments, in step (c) the level of accumulation measured in step (b) is compared with a predetermined level. In some embodiments, the method of treating cancer further comprising measuring the level of accumulation at a reference site and in step (c) the level of accumulation measured in step (b) is compared with the level of accumulation at the reference site. In some embodiments, the reference site is plasma, bone or muscle.

In another aspect of the invention, provided is a method of predicting the localization of NTA comprising:
(a) administering a contrast agent to a subject;
(b) conducting an imaging evaluation of the contrast agent at at least one intended site of treatment; and
(c) predicting the ability of the intended site of treatment to accumulate the NTA based on the accumulation of the contrast agent at the intended site of treatment.

In another aspect of the invention, provided is a method of assessing the efficacy of NTA in treating a subject with cancer comprising:
(a) administering a contrast agent to the subject before treatment with NTA,
(b) performing a pre-treatment imaging evaluation of regions of the subject's body targeted by NTA,
(c) administering NTA to the subject,
(d) administering the contrast agent to the subject after NTA treatment,
(e) performing a post-treatment imaging evaluation of the regions of the subject's body targeted by NTA treatment, and
(f) identifying any change in the post-treatment imaging evaluation compared to the pre-treatment imaging evaluation, wherein a decrease in the amount of contrast agent post-treatment in the targeted regions indicates the NTA is an effective treatment, and,
   wherein the regions of the subject's body targeted by NTA are tumor sites.

In another aspect of the invention, provided is a population of nanoparticles having PEG density of between about 0.04 units/nm² or 0.04 g/nm² and about 3.0 units/nm² or 3.0 g/nm², inclusive. In some embodiments, the average diameter of the nanoparticles is between 20 nm and 999 nm, inclusive. In some embodiments, the nanoparticles comprise a therapeutic agent. In some embodiments, the nanoparticles comprise a polymer or lipid or a combination thereof. In some embodiments, the nanoparticles comprise a surfactant or lyoprotectant or a combination thereof,

Other embodiments, objects, features, and advantages will be set forth in the detailed description of the embodiments that follow and, in part, will be apparent from the description or may be learned by practice of the claimed invention. These objects and advantages will be realized and attained by the compositions and methods described and claimed herein. The foregoing Summary has been made with the understanding that it is to be considered as a brief and general synopsis of some of the embodiments disclosed herein, is provided solely for the benefit and convenience of the reader, and is not intended to limit in any manner the scope, or range of equivalents, to which the appended claims are lawfully entitled.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIG. 1 is a graph showing nanoparticle tumor concentration is dependent on nanoparticle PEG density.

FIG. 2A shows macromolecule contrast agent AngioSense correlates with nanoparticle accumulation in various tumors.

FIG. 2B shows iron oxide nanoparticle contrast agent AngioSPARK correlates with nanoparticle accumulation in various tumors.

FIG. 3A shows images of AngioSPARK and Polymeric Nanoparticle D in A2780 ovarian cancer xenografts at 24 hours.

FIG. 3B is a merged image of the images of AngioSPARK and Polymeric Nanoparticle D in A2780 ovarian cancer xenografts at 72 hours.

FIG. 4 shows nanoparticle tumor concentration is dependent on tumor vasculature.

### DETAILED DESCRIPTION OF THE INVENTION

While the present disclosure is capable of being embodied in various forms, the description below of several embodiments is made with the understanding that the present disclosure is to be considered as an exemplification of the claimed subject matter, and is not intended to limit the appended claims to the specific embodiments illustrated and/or described, and should not be construed to limit the scope or breadth of the present invention. The headings used throughout this disclosure are provided for convenience only and are not to be construed to limit the claims in any way. Embodiments illustrated under any heading may be combined with embodiments illustrated under any other heading.

### I. Definitions

For convenience, before further description of the present teachings, certain terms employed in the specification, examples, and appended claims are collected here. These definitions should be read in light of the remainder of the disclosure and as understood by a person of ordinary skill in the art. Unless defined otherwise, all technical and scientific terms used herein have the same meaning as commonly understood by a person of ordinary skill in the art.

### A. General Terms

The articles “a” and “an,” as used herein, should be understood to mean “at least one,” unless clearly indicated to the contrary.

The phrase “and/or,” as used herein, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements).

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements.

In general, the term “or” as used herein shall only be interpreted as indicating exclusive alternatives (i.e. “one or the other but not both”) when preceded by terms of exclusivity, such as “either,” “one of,” “only one of,” or “exactly one of”.

As used herein, the phrase “at least one” in reference to a list of one or more elements should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it were individually recited herein.

### B. Terms Related to Drug Conjugates

The term “compound”, as used herein, is meant to include all stereoisomers, geometric isomers, tautomers, and isotopes of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H- 1,2,4-triazole, 1H- and 2H- isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

A “target” shall mean a site to which targeted constructs bind. A target may be either in vivo or in vitro. In certain embodiments, a target may be cancer cells found in leukemias or tumors (e.g., tumors of the brain, lung (small cell and non-small cell), ovary, prostate, breast and colon as well as other carcinomas and sarcomas). In other embodiments, a target may be a site of infection (e.g., by bacteria, viruses (e.g., HIV, herpes, hepatitis)) and pathogenic fungi (e.g., Candida sp.). Certain target infectious organisms include those that are drug resistant (e.g., Enterobacteriaceae, Enterococcus, Haemophilus influenza, Mycobacterium tuberculosis, Neisseria gonorrhoeae, Plasmodium falciparum, Pseudomonas aeruginosa, Shigella dysenteriae, Staphylococcus aureus, Streptococcus pneumoniae). In still other embodiments, a target may refer to a molecular structure to which a targeting moiety or ligand binds, such as a hapten, epitope, receptor, dsDNA fragment, carbohydrate or enzyme. Additionally, a target may be a type of tissue, e.g., neuronal tissue, intestinal tissue, pancreatic tissue etc.

“Target cells,” which may serve as the target for the method or coordination complexes of the present invention, include prokaryotes and eukaryotes, including yeasts, plant cells and animal cells. The present method may be used to modify cellular function of living cells in vitro, i.e., in cell culture, or in vivo, in which the cells form part of or otherwise exist in plant tissue or animal tissue. Thus, the target cells may include, for example, the blood, lymph tissue, cells lining the alimentary canal, such as the oral and pharyngeal mucosa, cells forming the villi of the small intestine, cells lining the large intestine, cells lining the respiratory system (nasal passages/lungs) of an animal (which may be contacted by inhalation of the subject invention), dermal/epidermal cells, cells of the vagina and rectum, cells of internal organs including cells of the placenta and the so-called blood/brain barrier, etc.

The term “cell” is understood to mean embryonic, fetal, pediatric, or adult cells or tissues, including but not limited to, stem cells, precursors cells, and progenitor cells. Examples of cells include but are not limited to immune cell, stem cell, progenitor cell, islet cell, bone marrow cells, hematopoietic cells, tumor cells, lymphocytes, leukocytes, granulocytes, hepatocytes, monocytes, macrophages, fibroblasts, neural cells, mesenchymal stem cells, neural stem cells, or other cells with regenerative properties and combinations thereof.

“Targeting ligand” or “targeting moiety” are used interchangeably and shall include a peptide, antibody mimetic, nucleic acid (e.g. aptamer), polypeptide (e.g. antibody), glycoprotein, small molecule, carbohydrate, or lipid.

As used herein, the term “linker” refers to a carbon chain that can contain heteroatoms (e.g., nitrogen, oxygen, sulfur, etc.) and which may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 atoms long. Linkers may be substituted with various substituents including, but not limited to, hydrogen atoms, alkyl, alkenyl, alkynl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. Those of skill in the art will recognize that each of these groups may in turn be substituted. Examples of linkers include, but are not limited to, pH-sensitive linkers, protease cleavable peptide linkers, nuclease sensitive nucleic acid linkers, lipase sensitive lipid linkers, glycosidase sensitive carbohydrate linkers, hypoxia sensitive linkers, photo-cleavable linkers, heat-labile linkers, enzyme cleavable linkers (e.g., esterase cleavable linker), ultrasound-sensitive linkers, x-ray cleavable linkers, and so forth.

The terms “therapeutic agent” or “active agent” or “pharmaceutically active agent” are art-recognized and refer to an agent capable of having a desired biological effect on a host.

The term “nanoparticle” as used herein refers to a particle having a characteristic dimension of less than about 1 micrometer, where the characteristic dimension of a particle is the diameter of a perfect sphere having the same volume as the particle. In general, the morphology of a nanoparticle has sphere-like properties or is spherical. The plurality or population of particles can be characterized by an average diameter (e.g., the average diameter for the plurality of particles). In some embodiments, the diameter of the particles may have a Gaussian-type distribution. In some embodiments, the plurality or population of nanoparticles have an average diameter of between 1 nm and 999 nm. In some embodiments, the plurality or population of particles have an average diameter of less than about 300 nm, less than about 250 nm, less than about 200 nm, less than about 150 nm, less than about 100 nm, less than about 50 nm, less than about 30 nm, less than about 10 nm, less than about 3 nm, or less than about 1 nm. In some embodiments, the particles have an average diameter of at least about 5 nm, at least about 10 nm, at least about 30 nm, at least about 50 nm, at least about 100 nm, at least about 150 nm, or greater. In certain embodiments, the plurality or population of the particles have an average diameter of about 10 nm, about 25 nm, about 50 nm, about 100 nm, about 150 nm, about 200 nm, about 250 nm, about 300 nm, about 500 nm, or the like. In some embodiments, the plurality or population of particles have an average diameter between about 10 nm and about 500 nm, between about 50 nm and about 400 nm, between about 100 nm and about 300 nm, between about 150 nm and about 250 nm, between about 175 nm and about 225 nm, or the like. In some embodiments, the plurality or population of particles have an average diameter between about 10 nm and about 500 nm, between about 20 nm and about 400 nm, between about 30 nm and about 300 nm, between about 40 nm and about 200 nm, between about 50 nm and about 175 nm, between about 60 nm and about 150 nm, between about 70 nm and about 120 nm, or the like. For example, the average diameter can be between about 70 nm and 120 nm.

C. Terms Related to Methods of Treatment and/or Assessment of Treatment

As used herein, a “subject” or a “patient” refers to any mammal (e.g., a human), such as a mammal that may be susceptible to a disease or disorder, for example, tumorigenesis or cancer. Examples include a human, a non-human primate, a cow, a horse, a pig, a sheep, a goat, a dog, a cat, or a rodent such as a mouse, a rat, a hamster, or a guinea pig. In various embodiments, a subject refers to one that has been or will be the object of treatment, observation, or experiment. For example, a subject can be a subject diagnosed with cancer or otherwise known to have cancer or one selected for treatment, observation, or experiment on the basis of a known cancer in the subject.

As used herein, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to reducing the progression of a disease or disorder, either physically, e.g., stabilization of a discernible symptom, physiologically, e.g., stabilization of a physical parameter, or both. In yet another embodiment, “treatment” or “treating” refers to delaying the onset of a disease or disorder.

As used herein, “prevention” or “preventing” refers to a reduction of the risk of acquiring a given disease or disorder.

The phrase “therapeutically effective amount” as used herein means that amount of a compound, material, or composition comprising a compound of the present teachings which is effective for producing some desired therapeutic effect. Accordingly, a therapeutically effective amount treats or prevents a disease or a disorder. In various embodiments, the disease or disorder is a cancer.

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human.

The term “modulation” is art-recognized and refers to up regulation (i.e., activation or stimulation), down regulation (i.e., inhibition or suppression) of a response, or the two in combination or apart.

As used herein, the terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, melanoma and various types of head and neck cancer.

“Tumor” and “neoplasm” as used herein refer to any mass of tissue that result from excessive cell growth or proliferation, either benign (noncancerous) or malignant (cancerous) including pre-cancerous lesions.

“Metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

As used herein, the term “subject” refers to any animal (e.g., a mammal), including, but not limited to humans, non-human primates, rodents, and the like, which is to be the recipient of a particular treatment. Typically, the terms “subject” and “patient” may be used interchangeably herein in reference to a human subject.

The terms “cancer cell”, “tumor cell” and grammatical equivalents refer to the total population of cells derived from a tumor including both non-tumorigenic cells, which comprise the bulk of the tumor cell population, and tumorigenic cells.

As used herein, “assessing stage of cancer” or “staging cancer” refers to any MRI information that is useful in determining whether a patient has a primary cancer or tumor, and/or metastatic cancer or tumor, and/or information that is useful in classifying the stage of the cancer into a phenotypic category or any category having significance with regards to the prognosis of or likely response to anticancer treatment (either anticancer treatment in general or any particular anticancer treatment) of the primary or metastatic tumor(s). Similarly, assessing stage of cancer refers to providing any type of information, including, but not limited to, whether a subject is likely to have a condition (such as a tumor), and information related to the nature or classification of a tumor as for example a high risk tumor or a low risk tumor, information related to prognosis and/or information useful in selecting an appropriate treatment. Selection of treatment can include the choice of a particular chemotherapeutic agent or other treatment modality such as surgery or radiation or a choice about whether to withhold or deliver therapy.

As used herein, the terms “providing a prognosis”, “prognostic information”, or “predictive information” refer to providing information regarding the impact of the presence of cancer (e.g., as determined by the staging methods of the present invention) on a subject's future health (e.g., expected morbidity or mortality, the likelihood of getting cancer, and the risk of metastasis).

The terms “systemic administration,” “administered systemically,” “peripheral administration” and “administered peripherally” are art-recognized and refer to the administration of a composition, therapeutic or other material other than directly into the central nervous system, such that it enters the patient's system and, thus, is subject to metabolism and other like processes, for example, intravenous or subcutaneous administration.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection.

### D. Terms Related to Pharmaceutics and Diagnostics

The term “pharmaceutically acceptable counter ion” refers to a pharmaceutically acceptable anion or cation. In various embodiments, the pharmaceutically acceptable counter ion is a pharmaceutically acceptable ion. For example, the pharmaceutically acceptable counter ion is selected from citrate, matate, acetate, oxalate, chloride, bromide, iodide, nitrate, sulfate, bisulfate, phosphate, acid phosphate, isonicotinate, acetate, lactate, salicylate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)). In some embodiments, the pharmaceutically acceptable counter ion is selected from chloride, bromide, iodide, nitrate, sulfate, bisulfate, phosphate, acid phosphate, citrate, matate, acetate, oxalate, acetate, and lactate. In particular embodiments, the pharmaceutically acceptable counter ion is selected from chloride, bromide, iodide, nitrate, sulfate, bisulfate, and phosphate.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, matate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

A pharmaceutically acceptable salt can be derived from an acid selected from 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, hydrochloric acid, isethionic, isobutyric acid, lactic acid, lactobionic acid, lauric acid, maleic acid, malic acid, malonic acid, mandelic acid, methanesulfonic acid, mucic, naphthalene-1,5-disulfonic acid, naphthalene-2-sulfonic acid, nicotinic acid, nitric acid, oleic acid, oxalic acid, palmitic acid, pamoic acid, pantothenic, phosphoric acid, proprionic acid, pyroglutamic acid, salicylic acid, sebacic acid, stearic acid, succinic acid, sulfuric acid, tartaric acid, thiocyanic acid, toluenesulfonic acid, trifluoroacetic, and undecylenic acid.

The term “bioavailable” is art-recognized and refers to a form of the subject invention that allows for it, or a portion of the amount administered, to be absorbed by, incorporated to, or otherwise physiologically available to a subject or patient to whom it is administered.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the formulation and not injurious to the patient.

As used herein, the term “in vitro” refers to events that occur in an artificial environment, e.g., in a test tube or reaction vessel, in cell culture, etc., rather than within an organism (e.g. animal, plant, and/or microbe).

As used herein, the term “in vivo” refers to events that occur within an organism (e.g. animal, plant, and/or microbe).

As used herein the terms “diagnose” or “diagnosis” or “diagnosing” refers to distinguishing or identifying a disease, syndrome or condition or distinguishing or identifying a person having a particular disease, syndrome or condition.

The term “diagnostic” refers to identifying the presence or nature of a pathologic condition. Diagnostic methods differ in their sensitivity and specificity.

The term “imaging agent” may be used interchangeably with “contrast agent” and refers to a compound that is capable of localizing selectively at sites of diagnostic interest in vivo, such as at a particular organ, tissue or cell type, to enhance imaging.

### II. Co-Localization of a Contrast Agent and NTA; Methods of Treating Disease and Condition and Assessing the Efficacy Thereof

Applicants have discovered that a contrast agent, e.g., a small electron dense moiety (EDM) such as an iron oxide containing particle, can be used to predict whether a polymeric nanoparticle, e.g., a nanoparticle therapeutic agent (NTA) that is larger than the contrast agent, is likely to accumulate in a tumor. A patient having a tumor can be assessed for whether a nanoparticle treatment with NTA is likely to be effective without labeling the NTA, thereby providing useful information in making treatment decisions.

A contrast agent as used herein is a molecule that can provide an image in an organism, e.g., an improvement or enhancement of an image in the body. A contrast agent may include an entity that has metallic properties (e.g., gadolinium, iron, indium etc.), semi-metallic properties (e.g., boron) or non-metallic properties (e.g. iodine). In some embodiments, a contrast agent can be radioactive or have magnetic properties. A contrast agent can be a nanoparticle (e.g., quantum dots such as cadmium selenide) or be part of a nanoparticle configuration in which the contrast agent is either incorporated, attached or both to the nanoparticle. In some embodiments, the contrast agent may provide a therapeutic effect.

Surprisingly, notwithstanding the size difference between an NTA and a contrast agent such as an imaging agent (e.g., an NTA may be about four times larger), differences in density, surface charge and composition, the NTA is able to localize and accumulate in the same target sites as the contrast agent, hereinafter called a ‘co-localization’ effect. Also, the level of accumulation of the NTA is generally proportional to the level of the accumulation of the contrast agent. The term “size”, as used herein, is characterized by the diameter of the particles of a contrast agent or NTA. The term “density” as used herein, means the quantity of mass per unit volume. The term “accumulation”, or “uptake”, or “localization”, used interchangeably, are used herein to describe the preferential of accumulation of nanoparticles at a target site, e.g., a tumor site, compared to the accumulation of nanoparticles at a reference site, e.g., plasma, bone or muscle. Without committing to any particular theory, contributing factors to the accumulation of a contrast agent and nanoparticles in a tumor are related to EPR and/or macrophage accumulation at a tumor site. The accumulation or uptake or localization of contrast agents or nanoparticles such as NTA may be detected with an imaging technique using a diagnostic device. The level of accumulation or uptake or localization of contrast agents or nanoparticles such as NTA may be characterized by tumor concentration of the contrast agents or nanoparticles such as NTA and may be measured by an imaging technique with a diagnostic device. The detection of the accumulation of contrast agents or nanoparticles such as NTA or measurement of the level of the accumulation of contrast agents or nanoparticles such as NTA is referred to as imaging evaluation of contrast agents and nanoparticles such as NTA. Imaging evaluation may be performed with a diagnostic device after administering a contrast agent to a subject. In some embodiments, the diagnostic device may be an ultrasound, fluorescence spectrometer, X-ray, Mill scanner, PET scanner, fluorescence tomography or CT scanner. In some embodiments, the target site of NTA is a tumor site. In some embodiments, the NTA targets malignant cells, non-malignant cells, or cancer stem cells at the tumor site.

The level of accumulation of a contrast agent is determined using any suitable method. The level of accumulation may be determined by comparing the signal from a site of interest, e.g., a tumor, to a reference. The reference can be predetermined or determined at the same time as the site of interest signal is acquired. In some embodiments, the level of accumulation can be determined by assaying the intensity of the signal originating from the contrast agent at the imaging site. This signal is then adjusted based on the concentration of the contrast agent used to yield a normalized signal. This can then be further quantified based on the amount of material imaged (e.g., weight of tumor tissue). The level of accumulation of the contrast agent can then be compared directly with an area that has a low level (background) of accumulation (e.g., muscle or plasma).

In some embodiments, the site of interest may be assayed at a specified time point, a time point associated with maximum accumulation of the contrast agent (defined as largest amount of contrast agent detected over a period of time) at a specific time point. In some embodiments, the contrast agent is detected at a time that is not that of maximal accumulation. In some embodiments, the contrast agent is detect at e.g., 1 hour, 2 hours, 3 hours, 4 hours, 5 hours, 8 hours, 10 hours, 15 hours, 20 hours 24 hours, 48 hours, 3 days, 4 days, 5 days, or 7 days after administration. In some cases, the reference used is the amount of contrast agent in plasma. For example, a standard volume of plasma is prepared and assayed. Alternatively the accumulation could be compared to a predetermined reference (e.g., a low level of accumulation) or a specified amount/tumor tissue at the site.

The present invention relates to a method for using a contrast agent, such as fluorescent macromolecules (e.g., AngioSense®), iron oxide nanoparticles (e.g., AngioSPARK®, Feraheme®) or other imaging agent, to establish whether a patient has a sufficient accumulation effect (e.g., EPR effect) for the subsequent administration of an NTA. The present invention also relates to the in vivo diagnosis, assessment and/or monitoring of disease progression either before or following treatment with a nanoparticle therapeutic agent or NTA.

Also, the level of accumulation of the NTA is generally proportional to the level of the accumulation of the contrast agent. This is unexpected in view of the fact that the size of a contrast agent is generally smaller than an NTA.

In some embodiments, the contrast agent and the NTA differ from one another based on at least one parameter by at least 2 folds. In some embodiments, the parameters are size, density, or surface charge.

In one embodiment, the contrast agent may be 2-100 times smaller than the NTA. In some embodiments, the contrast agent may be 2-50 times smaller than the NTA. In some embodiments, the contrast agent may be 2-10 times smaller than the NTA. In some embodiments, the contrast agent is 3-6 times smaller than the NTA. In a further embodiment, the contrast agent consists of two monodispersed particle size ranges. In such embodiments, the smaller particle within the contrast agent is 6-10 times smaller than the NTA, and the larger particle within the contrast agent will be 3-5 times smaller than the NTA. In some embodiments, the size of a contrast agent is between about 1 nm and about 15 nm. In some embodiments, the size of a contrast agent is between about 1 nm and about 10 nm. In some embodiments, the size of a contrast agent is between about 1 nm and about 6 nm. In some embodiments, the size of an NTA is between about 20 nm and about 999 nm. In some embodiments, the size of an NTA is between about 20 nm and about 200 nm. In some embodiments, the size of a contrast agent is about 30 nm and an NTA is about 100 nm. The term “smaller”, as used herein, means the diameter of the contrast agent is smaller than the diameter of the NTA.

In one aspect of the invention, a method of identifying or selecting patients that will benefit from NTA therapy is provided. A contrast agent is used to identify patients whose tumors (i.e., one or more of their tumors) have an accumulation effect, e.g., EPR effect, that is sufficiently robust to allow accumulation of sufficient amount of NTA. To facilitate such identification, the patient is administered with a sufficient amount of the contrast agent and the accumulation of the contrast agent at tumor sites will be used to assess the accumulation effect in that particular treatment. The accumulation effect may be based on the intensity of the signal within the tumor or the area coverage within the tumor. The robustness of accumulation may be determined by comparing the accumulation of the contrast agent at the tumor sites to a reference (e.g., muscle or plasma), or to a predetermined level of accumulation. For example, a subject may have robustness of accumulation if the accumulation of the contrast agent at the tumor site is higher than at a reference site.

In another embodiment, the decision to treat patients using the NTA therapy will be made on the basis of the robustness of the accumulation effect as established by the contrast agent as described above. For example, patients may be classified into designated groups to aid in the treatment decision-making algorithm, e.g., low-accumulation, medium-accumulation, and high-accumulation. Such classifications may be based on signal intensity and amount of area coverage of the contrast agent. For example, a particular contrast agent's uptake may be classified as: Excellent (more than about 90%), good (about 70%-90%), moderate (about 50%-70%), low (about 30%-50%) or poor (less than about 30%) of the contrast agent's localization at the target site. In another example, the extent of accumulation may be correlated to the expected toxicity or efficacy of a drug. In some embodiments, data of level of accumulation may be collected from a number of patients with specific disease types and comparing them as a whole. Low and high accumulation boundaries could be established based on the patients (assuming there is a diverse patient population that responds to the contrast agent).

In another embodiment, in patients with multiple tumor sites, assessment of the robustness at each of the tumor sites may be performed separately. In a further embodiment, a decision to treat a patient using the NTA therapy may be based on the robustness of accumulation of one or more specific tumor sites. For example, NTA therapy as neo-adjuvant therapy may be used to shrink the tumor at specific sites prior to surgical resection.

The tumor environment is dynamic and factors affecting accumulation of a contrast agent such as the EPR effect may change. Non-limited examples of factors affecting the EPR effect in solid tumors are disclosed in on page 3 and Table 1 of Prabhakar et al., Cancer Res., vol. 73(8):2412-2417 (2013), the contents of which are incorporated herein by reference in their entireties. In one embodiment, the contrast agent may be used iteratively at different times to assess the accumulation effect in the tumor environment. In a further embodiment, the repeated measure of accumulation may be used to adjust the course of the NTA therapy. For example, a patient that is not initially selected for NTA therapy may later show robust accumulation and in view of the robust accumulation, be prescribed and administered NTA therapy.

In a further embodiment, the assessment of the EPR effect may be tied to treatment with agents that modulate the EPR effect. See, e.g., H. Maeda, “Macromolecular therapeutics in cancer treatment: The EPR effect and beyond,” J. Controlled Release 164: 138-44 (2012), the contents of which are incorporated herein by reference in their entirety. Any EPR modulating agents disclosed by Maeda may be used. In a further embodiment, accumulation modulating agents may be administered to patients that may not have originally been a candidate for NTA therapy to increase the accumulation effect in such patients.

In addition to the absolute amount of NTA at the tumor site, the relative amount of NTA in the tumor as compared to another non-tumor tissue may have an impact on the balance between efficacy and toxicity for the NTA. As an example, for an NTA that exhibits neural toxicity by accumulating in the dorsal root ganglion, it may be important to understand in an individual patient the relative retention of nanoparticles at the tumor site as compared to the dorsal root ganglion. One aspect of the invention provides for selecting patients for NTA based on predicted distribution of nanoparticles between a site and a non-target site. In one embodiment, the assessment of the accumulation effect using the contrast agent is used to predict the relative distribution between the two sites.

In other embodiments, the present invention relates to methods for using contrast agents for the in vivo monitoring and assessment of disease progression following treatment with an NTA. In one embodiment, the method comprises: administering a contrast agent; establishing a pre-treatment image of the subject's body to be targeted by the NTA with a diagnostic device; administering the NTA; administering a contrast agent following treatment with the drug conjugate establishing a post-treatment image of the subject's body targeted by the drug conjugate; and assessing any change in the post-treatment image compared to the pre-treatment image with respect to disease progression. In one embodiment, the diagnostic device is an ultrasound, X-ray, MRI scanner, PET scanner or CT scanner.

In a further embodiment, a method is provided for treating a disease or condition with a drug conjugate, the method comprising administering a diagnostic imaging agent; establishing a pre-treatment image of the subject's body to be targeted by the drug conjugate; administering a therapeutically effective amount of the drug conjugate NTA; administering a diagnostic imaging agent following treatment with the drug conjugate; establishing a post-treatment image of the subject's body targeted by the drug conjugate; assessing any change in the post-treatment image compared to the pre-treatment image with respect to disease progression; and repeating as needed. In some embodiments, the NTA may be a drug conjugate or contain a drug conjugate that has been found to inhibit one or more features of cancer growth, including hyperproliferation, invasiveness, and metastasis, thereby rendering the NTA particularly desirable for the treatment of cancer. In some embodiments, the drug conjugates may be used to shrink or destroy a cancer. The method allows assessment of the drug conjugate by comparing imaging evaluation before treatment, between treatment cycles, and after treatment of the drug conjugate. In one aspect, the disease is a cancer or hyperproliferative disease, including but not limited to brain cancer, cervical cancer, esophageal cancer, gallbladder cancer, head and neck cancer, kidney cancer, liver cancer, multiple myeloma, thyroid cancer, lymphoma, renal cell carcinoma, leukemia, prostate cancer, lung cancer, pancreatic cancer, melanoma, colorectal cancer, ovarian cancer, breast cancer, glioblastoma multiforme and leptomeningeal carcinomatosis.

In one embodiment, the methods include the use of a contrast agent, wherein the image includes observing accumulation activity of the contrast agent associated with a primary tumor or with any metastatic tumor in bone, lymph node, spleen, liver, central nervous system, lung, or other organ. In one embodiment, the regions collectively include the entire body. In other embodiments, the contrast agent is an ultrasmall superparamagnetic iron oxide particle, and in still more embodiments, the contrast agent has a blood half-life sufficient to permit microphage trapping throughout the regions at cancer risk. In yet another embodiment, the contrast agent is a complex of ultrasmall superparamagnetic iron oxide and a polysaccharide. In still other embodiments, the polysaccharide is selected from the group consisting of dextrans, reduced dextrans and a derivative thereof.

Another embodiment provides a method for determining the prognosis of cancer in a subject following treatment with an NTA, the method comprising assessing any change in the post-treatment image compared to the pre-treatment image with respect to contrast agent level of accumulation and displacement associated with a primary cancer or metastatic cancer in the subject. The prognosis of cancer in the subject is based on level of accumulation of the contrast agent at the primary and/or metastatic tumors, the level of accumulation being an indicator of the prognosis of the cancer whereby low level of accumulation relative to normal cells is an indicator of a more favorably prognosis and high level of accumulation relative to normal cells is an indicator of a less favorable prognosis.

Another particular embodiment provides a method for providing individualized cancer treatment to a subject in need thereof using imaging evaluation, the method comprising performing a pre-treatment imaging evaluation of the subject to identify level of accumulation of a contrast agent at a primary and/or tumor site of interest, assessing the level of accumulation to identify characteristics (type, location, phenotypic and morphological) of the primary and/or metastatic tumors in the subject, assessing the characteristics of the primary and/or metastatic tumors in the subject to determine the optimal treatment with a NTA, administering the NTA, performing a post-treatment imaging evaluation of the subject to determine level of accumulation of a contrast agent at the primary and/or tumor site of interest, assessing the level of accumulation to identify characteristics (type, location, phenotypic and morphological) of the primary and/or metastatic tumors in the subject, assessing the characteristics of the primary and/or metastatic tumors in the subject, and providing individualized cancer treatment to the subject based on the assessment of the primary and/or metastatic tumors in the subject prior to and post-treatment with the NTA as determined using imaging evaluation.

The cancers treatable by methods of the present teachings preferably occur in mammals. Mammals include, for example, humans and other primates, as well as pet or companion animals, such as dogs and cats, laboratory animals, such as rats, mice and rabbits, and farm animals, such as horses, pigs, sheep, and cattle. In various embodiments, the cancer is lung cancer, breast cancer, colorectal cancer, ovarian cancer, bladder cancer, prostate cancer, cervical cancer, renal cancer, leukemia, central nerve system cancers, myeloma, and melanoma. In some embodiments, the cancer is lung cancer. In certain embodiments, the cancer is human lung carcinoma and/or normal lung fibroblast.

Other diseases besides cancer may also be treated and/or diagnosed with the NTA. Any disease that would benefit from the administration of an NTA could be treated and/or diagnosed with the disclosed method. Such diseases may include hyperproliferative diseases, cardiovascular diseases, gastrointestinal diseases, genitourinary disease, neurological diseases, musculoskeletal diseases, hematological diseases, inflammatory diseases, and autoimmune diseases.

### A. Contrast Agents

In some embodiments, contrast agents include gases; commercially available imaging agents used in positron emissions tomography (PET), computer assisted tomography (CAT), single photon emission computerized tomography, x-ray, fluoroscopy, and magnetic resonance imaging (MRI); antiemetics; and any other contrast agents. Examples of suitable materials for use as contrast agents in Mill include gadolinium chelates, as well as iron, magnesium, manganese, copper, and chromium. Examples of materials useful for CAT and x-ray imaging include iodine-based materials.

In some embodiments, the contrast agent may comprise a diagnostic agent used in magnetic resonance imaging (MRI), such as iron oxide particles or gadolinium complexes. Gadolinium complexes that have been approved for clinical use include gadolinium chelates with DTPA, DTPA-BMA, DOTA and HP-DO3A (reviewed in Aime et al., 1998, Chemical Society Reviews, 27:19). In another embodiment, the contrast agent used is ferrumoxitol. Some contrast agents that may be useful in carrying out the presently claimed invention are summarized in EP0502814B1, the contents of which are hereby incorporated by reference herein.

In some embodiments, a diagnostic agent may be a fluorescent, luminescent, radioactive, or magnetic moiety. In some embodiments, a detectable moiety such as a fluorescent or luminescent dye, etc., is entrapped, embedded, or encapsulated by a particle core and/or coating layer.

Fluorescent and luminescent moieties include a variety of different organic or inorganic small molecules commonly referred to as “dyes,” “labels,” or “indicators.” Examples include fluorescein, rhodamine, acridine dyes, Alexa dyes, cyanine dyes, etc. Fluorescent and luminescent moieties may include a variety of naturally occurring proteins and derivatives thereof, e.g., genetically engineered variants. For example, fluorescent proteins include green fluorescent protein (GFP), enhanced GFP, red, blue, yellow, cyan, and sapphire fluorescent proteins, reef coral fluorescent protein, etc. Luminescent proteins include luciferase, aequorin, and derivatives thereof. Numerous fluorescent and luminescent dyes and proteins are known in the art (see, e.g., U.S. Patent Publication 2004/0067503; Valeur, B., “Molecular Fluorescence: Principles and Applications,” John Wiley and Sons, 2002; Handbook of Fluorescent Probes and Research Products, Molecular Probes, 9^{th} edition, 2002; and The Handbook—A Guide to Fluorescent Probes and Labeling Technologies, Invitrogen, 10^{th} edition, available at the Invitrogen web site). Fluorescent molecules useful in the methods provided herein vascular imaging agents, for example, Angiospark® (a pegylated iron core fluorescent macromolecule) flu and AngioSense® (a pegylated poly-L lysine near-infrared labeled fluorescent macromolecule). AngioSense® may be used as a fluorescent in vivo blood pool imaging agent. It remains in the vasculature for extended periods of time and serves to provide details on the tumors that are investigated and how much the tumors are vascularized.

Non-limiting examples of contrast agents used to enhance imaging are compounds containing carbon-11, oxygen-15, nitrogen-13, and fluorine-18; compounds containing iodine-123, iodine-124, iodine-125, and iodine-131; compounds containing indium-111, mangafodipir trisodium, amidotrizoate, EVP 1001-1, iothalamate, ioxithalamate, ioxaglate, iohexol, iopentol, ioxilan, iomeprol, ioversol, iopromide, iobitridol, iopamidol, iotrolan, iodixanol, gadopentetate dimeglumine, gadodiamide, gadoversetamide, gadoterate dimeglumine, gadobutrol, gadoteridol, gadobenate dimelumine, gadofosveset trisodium, gadoxetate disodium, ferumoxytol (Feraheme®), ferumoxsil, ferristene, ferumoxides, ferucarbotran, ferumoxtran, ferric chloride, ferric ammonium citrate, and the like. More examples of contrast agents are found in US 20130038330, US 20120035434, US 20110104052, US 20100080788, and WO2011103182, the contents of which are incorporated here by reference.

In some embodiments, the contrast agent is iron oxide-based contrast agents. They significantly affect the contrast of the images even when used in very small amounts. In some embodiments, the contrast agent is ferrumoxytol, a superparamagnetic iron oxide nanoparticle coated with polyglucose sorbitol caboxymethylether. It is considered an ultrasmall superparamagnetic particles iron oxide particle. The coating of the particles delays their degradation, resulting in isolation of the bioactive iron from plasma components and creating a long-lived distribution following administration. Other advantages of using such magnetic nanoparticles are the bioavailability, the high level of accumulation as a specific site and the low toxicity effects. The use of ferumoxytol as an MRI contrast agent is undergoing clinical trials in various studies including “Ferumoxytol Enhanced MRI for the Detection of Lymph Node Involvement in Prostate Cancer” and “Ferumoxytol and Gadolinium Magnetic Resonance Imaging (MRI) at 3T and 7T in Patients With Malignant Brain Tumors.” See ClinicalTrials.gov; Identifier: NCT01296139 and NCT00659126, respectively.

The contrast agent may be administered by any route in an amount sufficient to be detected with a suitable imaging technique. In some embodiments, the contrast agent is administered orally, by injection, or intravenously. In some embodiments, the contrast agent is administered to a subject between about 12 to about 336 hours prior to imaging evaluation. In some embodiments, a contrast agent is administered to a subject between about 12 hours to about 168 hours prior to imaging evaluation.

### B. Diagnostic Methodologies to Assess Efficacy of a Drug Conjugate

In some embodiments, the method of the present invention may be used to monitor and assess the treatment efficacy of an NTA by conducting imaging evaluation of a contrast agent in a subject pre- and post-treatment, and assessing any change in the post-treatment image compared to the pre-treatment image with respect to disease progression. Another embodiment provides methods for characterizing and assessing cancer progression, growth and potential for and/or actual metastasis by conducting imaging evaluation of a contrast agent following treatment with a drug conjugate. The imaging evaluation may be whole or only a specific area of the body, such as a tumor site. The imaging evaluation may not be an actual image of the subject, but may be an analysis of signal received by a diagnostic device adapted to detect the contrast agent with an imaging technique.

A contrast agent is administered to a subject, and the subject is then imaged using a technique with the ability to detect the administered contrast agent. In certain embodiments, the imaging technique used is single-photon emission tomography/computed tomography (SPECT/CT). In certain embodiments, the imaging technique used is positron emission tomography/computed tomography (PET/CT). In certain embodiments, the imaging technique used is positron emission tomography (PET). In certain embodiments, the imaging technique used is magnetic resonance imaging (MRI). In certain embodiments, the imaging technique used is computed tomography (CT). In certain embodiments, the imaging technique used is single-photon emission tomography (SPECT). In certain embodiments, the imaging technique is fluorescence spectroscopy or fluorescence tomography. Any of the imaging techniques described herein may be used in combination with other imaging techniques.

In some embodiments, the imaging technique is Magnetic Resonance Imaging (MRI). MRI uses a uniform magnetic field and radio frequency pulses to produce contrast images of the organs and tissues within the body. The protons (¹H nuclei) of the water molecules present in the body tissues, align in the large magnetic field with the direction of the field. A radio frequency pulses are applied resulting in flipping of the spin of the protons. After the radio frequency is turned off, a re-alignment of the spin with the magnetic field takes place. There are two types of relaxation, the spin-spin relaxation (T₁) and spin-lattice relaxation times. Introducing an imaging agent affects the spin re-equilibration of the nuclei. They are referred to as positive MRI contrast agents if they affect T₁ relaxation time or negative MRI contrast agents if they affect T₂ relaxation time. Examples on the positive MRI contrast agent are the gadolinium-based contrast agents. Iron oxide-based (ferric oxide or ferroxide based) contrast agents are examples of negative MRI contrast agents.

Whole body MRI technology has been known and used for a number of years. For example, U.S. Pat. Nos. 6,963,768, 6,681,132, 6,975,113 and 7,227,359 describe non-limiting examples of methods and systems that can be used for performing continuous whole body MRI. Similarly, U.S. Pat. No. 7,738,944 and U.S. Publication No. also disclose whole body MRI methods and apparatus. One or more of the above-disclosed methodologies and apparatus may be useful to carry out various embodiments of the presently claimed invention. Accordingly, the entire contents of the above-referenced U.S. patents (U.S. Pat. Nos. 6,963,786; 6,681,132; 6,975,113; 7,227,359; 7,738,944) and US Published Application (20050154291) are hereby incorporated by reference herein in their entirety.

Traditional magnet systems for MRI scanners have to accommodate the insertion of a human being and generate a homogeneous region large enough to cover a cylindrical area with a diameter between about 20 to about 50 cm, preferably about 40 cm, spherical volume (DSV) over the subject. For sufficient image quality, the magnets are typically made from permanent magnets in low-field systems (<5,000 gauss; <0.5 T) and superconducting magnet systems in high field systems (>10,000 gauss; >IT). Nael et al. (2007) Am. J. Radiol, 188, 529-39, the contents of which are incorporated herein by reference in their entirety, shows an illustration of a patient placed within a whole-body MRI system for scanning with the use of contrast agents.

MRI uses nuclear magnetic resonance (NMR) to visualize internal features of a living subject, and is useful to produce for prognosis, diagnosis, treatment, and surgery. Generally, the differences related to relaxation time constants T1 and T2 of water protons in different environments are used to generate an image. However, these differences can be insufficient to provide sharp high resolution images with adequate depiction of health or disease.

The differences in the relaxation time constants can be enhanced by contrast agents, as described above. Examples of such contrast agents include a number of magnetic agents, such as paramagnetic agents (which primarily alter T1) and ferromagnetic or superparamagnetic (which disproportionately alter T2 response). Chelates (e.g., EDTA, DTPA and NTA chelates) can be used to attach (and reduce toxicity) of some paramagnetic substances (e.g., Fe⁺³, Mn⁺², Gd⁺³). Other agents can be in the form of particles, e.g., less than 10 μm to about 10 nM in diameter). Particles can have ferromagnetic, antiferromagnetic or superparamagnetic properties. Particles can include, e.g., magnetite (Fe3O4), gamma-Fe2O3, ferrites, and other magnetic mineral compounds of transition elements. Magnetic particles may include: one or more magnetic crystals with and without nonmagnetic material. The nonmagnetic material can include synthetic or natural polymers (such as sepharose, dextran, dextrin, starch and the like.

In some embodiments, the contrast agents are iron oxide nanoparticles. They have long blood half-life resulting in better macrophage accumulation. Contrast agents that may be used in embodiments of the presently claimed invention include but not limited to Feraheme and ferumoxtran-10. Ferahame and ferumoxtran-10 are MRI agents that are superparamagnetic, and fall within a class known as ultrasmall superparamagnetic iron oxide particles. In one study, useful iron oxide nanoparticles such as ferumoxtran-10 were studied for their effect on macrophages in vitro and found to be non-toxic to human monocyte-macrophages (Gillard et al., Biomaterials 28 (2007) 1629-1642). In general, ultrasmall superparamagnetic iron oxide particles that comprise polyols, polyethers and/or polysaccharides, particularly reduced polysaccharides, more particularly carboxyalkylated reduced polysaccharides, are useful for embodiments of the MRI scanning described here. In a particular embodiment, the polysaccharide of the ultrasmall superparamagnetic particles iron oxide particles is a carboxyalkylated reduced dextran iron oxide complex.

In one embodiment, the ultrasmall superparamagnetic particles are iron oxide containing particles, e.g., ferumoxytol (e.g., Feraheme®). Ferumoxytol is a non-stoichiometric magnetite (superparamagentic iron oxide) coated with polyglucose sorbitol carboxymethylether. The overall colloidal particle size is 17-31 nm with an apparent molecular weight of 750 kDa.

In some embodiments, MRI contrast agents useful for embodiments of the presently claimed invention may be rare macrophage-seeking agents, such as the ultrasmall superparamagnetic iron oxide particles disclosed in the following patents and applications, the contents of which are all hereby incorporated by reference herein in their entirety: U.S. Pat. No. 5,160,726 (Filter Sterilization for Production of Colloidal Superparamagnetic MR Contrast Agents); U.S. Pat. No. 5,262,176. (Synthesis of Polysaccharide Covered Superparamagnetic Oxide Colloids); U.S. Pat. No. 6,599,498 (Heat Stable Colloidal Iron Oxides Coated With Reduced Carbohydrates and Carbohydrate Derivatives); and U.S. Pat. No. 7,553,479 (Heat Stable Colloidal Iron Oxides Coated With Reduced Carbohydrates and Uses Thereof); and U.S. Pat. No. 7,871,597 (Polyol and Polyether Iron as Pharmacological and/or MRI Contrast Agents). In particular embodiments the contrast agent is used as a single contrast agent. In related embodiments, the contrast agent is used in combination with another contrast agent.

Administration of any one of a class of macrophage-seeking contrast agents followed by a MRI enables visualization of tissue surrounded by or associated with macrophages, which tissue will be enhanced in the MR image by the macrophage-seeking contrast agent. This in turn permits, inter alia, an assessment of anticancer therapy, by comparison of tumor number, size, morphology and location, among other characteristics, observed with MRI before treatment, between treatment cycles and after the anticancer treatment.

Using macrophage-seeking contrast agents and MRI to perform a MRI evaluation as described above allows a physician to (a) provide a more accurate assessment of the metastatic potential of the primary tumor, (b) determine the degree of metastasis that may have already begun, (c) identify the location of the metastatic tumors, (d) customize the drug conjugate based on the characteristics and metastatic extent of the primary tumor (or metastatic tumors already present), and (e) assess the efficacy of such treatment.

### C. Therapeutic Nanoparticles

In one aspect, any nanoparticle therapeutic agent can be utilized in the methods of the present invention. In some embodiments, the NTA can be a nanoparticle drug conjugate. As a non-limiting example, the nanoparticle drug conjugate can be a triple-targeted nanoparticle drug conjugate as described in the U.S. Provisional Application No. 61/746,866, PCT/US13/78361, 62/019,001, 62/019,003, and 62/020,615, the contents of which are incorporated herein by reference in their entireties, which provides methods for active molecular targeting employing a bioactivated prodrug with accumulation effect and improved biodistribution. Without limiting the teachings of the disclosure, “triple-targeted” refers to a nanoparticulate composition comprising (1) one or more targeting ligands that bind to a target cell; (2) one or more pharmaceutically active agents linked in a prodrug form to the ligand that treats or modulates a disease or condition at the target cell; and (3) at least one polymer encapsulating all or part of the conjugate of the active agent and the ligand, wherein due to the an accumulation effect, e.g., EPR effect, the nanoparticle accumulates in the target tissue to be differentially retained while the active agent is released.

One embodiment includes a nanoparticle, comprising an inner portion and an outer surface, the inner portion comprising a conjugate of a targeting ligand and an active agent connected by a linker, wherein the conjugate has the formula:
(X—Y—Z)
wherein:
X is a targeting ligand;
Y is a linker; and
Z is a pharmaceutically active agent.

In another embodiment, one ligand may be conjugated to two or more active agents wherein the conjugate has the formula: X—(Y—Z)ₙ. In a further embodiment, one active agent molecule may be linked to two or more ligands wherein the conjugate has the formula: (X—Y)ₙ—Z. n is an integer equal to or greater than 1.

In one embodiment, X can be a peptide, antibody mimetic, nucleic acid (e.g. aptamer), polypeptide (e.g. antibody or its fragment), glycoprotein, small molecule, carbohydrate, or lipid. In another embodiment, X can be a peptide such as somatostatin, octeotide, EGF or RGD-containing peptides; an aptamer being either RNA or DNA or an artificial nucleic acid; small molecules; carbohydrates such as mannose, galactose and arabinose; vitamins such as ascorbic acid, niacin, pantothenic acid, carnitine, inositol, pyridoxal, lipoic acid, folic acid (folate), riboflavin, biotin, vitamin B₁₂, vitamin A, E, and K; a protein such as thrombospondin, tumor necrosis factors (TNF), annexin V, interferons, angiostatin, endostatin, cytokines, transferrin, GM-CSF (granulocyte-macrophage colony-stimulating factor), or growth factors such as vascular endothelial growth factor (VEGF), hepatocyte growth factor (HGF), platelet-derived growth factor (PDGF), basic fibroblast growth factor (bFGF), and epidermal growth factor (EGF).

In yet another embodiment, X can be RGD peptide, folic acid or prostate specific membrane antigen (PSMA).

In various aspects, Y is a linker bound to an active agent and a targeting ligand to form a conjugate wherein the conjugate releases at least one active agent upon delivery to a target cell.

In one embodiment, Z can be a chemotherapeutic agent, antibiotic, antimicrobial, growth factor and combinations thereof. In another aspect, Z may be cabazitaxel, a platinum(IV) complex, or analogues or derivatives thereof.

The conjugates taught herein may be formulated as nanoparticles such as, for example, liposomes, nanosuspensions, polymeric nanoparticles, dendrimers, fullerenes, carbon nanotubes, and inorganic nanoparticles. In some embodiments they are encapsulated, in whole or in part, in the inner portion of the nanoparticles. The nanoparticles may have a substantially spherical or non-spherical configuration (e.g., upon swelling or shrinkage). The nanoparticles may include polymer blends. In various embodiments, the base component of the nanoparticles comprises a polymer, a small molecule, or a mixture thereof. The base component can be biologically derived. For example, the small molecule can be, for example, a lipid. A “lipid,” as used herein, refers to a hydrophobic or amphiphilic small molecule. Without attempting to limit the scope of the present teachings, lipids, because of their amphiphilicity, can form particles, including liposomes and micelles. In one embodiment, any therapeutic nanoparticle having accumulation effect can be useful in the methods disclosed herein, including the compositions disclosed in the following U.S. patents and applications owned or licensed by Applicant, which are incorporated herein by reference in their entireties:

| Pats. | application Ser. Nos. | Publications |
|---|---|---|
| 6,254,890 | U.S. 13/468,477 | US2011027172 |
| 7,651,979 | U.S. Pat. No. 61,739,234 | US2011257261 |
| | U.S. 61/746,866 | US2011300219 |
| | U.S. 61/791,109 | US2012156135 |
| | U.S. 61/792,665 | US2013029959 |
| | U.S. 62/019,001 | US2011065807 |
| | U.S. 62/020,615 | US2013017265 |
| | U.S. 62/019,003 | WO2014106208 |
| | | US20140187501 |

The following agents may also be useful in the methods disclosed herein: Abraxane®, Doxil®, Daunoxome®, Depocyt®, Marqibo®, Genexol® PM, Nanotherm®, Myocet®, Nanoxel, MM-398 (Merrimack Pharmaceuticals), Lipoplatin®, Lipoxal, NK-105, Nanoplatin®, NK-4016, MBP-426, CRLX-101, CRLX-301, MM-302, CPX-351, CPX-1, CPX-571, SLIT Cisplatin, LEP-ETU, Thermodox®, SP-1049c, CALAA-01, Cyt-6091, Aurolase, Livatag®, Paclical, LiPlaCis, and SACN.

Other examples of nanoparticulate compositions useful in the present invention include those described in the following U.S. Patents and applications, which are incorporated herein by reference in their entireties: U.S. Pat. No. 8,329, 213; 2013122056; U.S. Pat. No. 8,475,781; 2013164400; U.S. Pat. No. 8,323,696; 2012029062; U.S. Pat. No. 8,211,656; 8,454,966; U.S. Pat. No. 7,270,808; 2013138032; U.S. Pat. No. 8,447,379; 2013011333; 2013115192; and 2013101672. Any nanomedicine disclosed in Table 2 of Prabhakar et al., Cancer Res., vol. 73(8):2412-2417 (2013), the contents of which are incorporated herein by reference in their entirety, may also be used in the methods disclosed herein.

### III. Methods of Modulating Tumor Concentration of Nanoparticles

The present invention relates to a method for modulating tumor concentration of nanoparticles such as NTA. Tumor concentration of NTA may affect the efficacy of NTA treatment. It is expected that increasing tumor concentration of NTA improves the efficacy of NTA treatment. Nanoparticle tumor concentration, as used herein, refers to the amount of nanoparticles at a tumor site.

In some embodiments, tumor concentration of NTA is modulated comprising controlling PEG density of the nanoparticles. Tumor concentration of NTA has a positive and statistically significant correlation with the PEG density of the nanoparticles. For example, tumor concentration increases with PEG density of the nanoparticles in highly vascularized tumor. Highly vascularized tumor, as used herein, refers to a tumor having adequate supply of blood from blood vessels. Methods of evaluating tumor vascularization are known in the art and may include, for example but not limited to, tumor vascularity measured by intercapillary distance (ICD), microvessel density (MVD), and tumor hypoxia. The term “PEG density”, as used herein, refers to the amount of PEG of the nanoparticles. It may be characterized with the mass of PEG or the number of PEG chains.

In some embodiments, nanoparticle PEG density is at least about 0.04 units/nm², 0.05 units/nm², 0.075 units/nm², 0.1 units/nm², 0.2 units/nm², 0.3 units/nm², 0.4 units/nm², 0.5 units/nm², 0.6 units/nm², 0.7 units/nm², 0.8 units/nm²,1.0 units/nm², 1.5 units/nm², 2.0 units/nm², 2.5 units/nm², or 3.0 units/nm². In some embodiments, the PEG density is from about 0.3 units/nm² to about 0.8 units/nm², inclusive The term “unit”, as used herein, refers to the number of PEG chains.

In some embodiments, nanoparticle PEG density is at least about 0.04 g/nm², 0.05 g/nm², 0.075 g/nm², 0.1 g/nm², 0.15 g/nm², 0.2 g/nm², 0.25 g/nm², 0.3 g/nm², 0.35 g/nm², 0.4 g/nm², 0.45 g/nm², 0.5 g/nm², 0.55 g/nm², 0.6 g/nm², 0.7 g/nm², 0.8 g/nm², 1.0 g/nm², 1.5 g/nm², 2.0 g/nm², 2.5 g/nm², or 3.0 g/nm². In some embodiments, PEG density is from about 0.3 g/nm² to about 0.8 g/nm².

In some embodiments, nanoparticles may be labeled with a fluorescence dye and tumor concentration of NTA is measured by fluorescence.

In some embodiments, NTA comprising at least one PEG moiety and a PEG density of at least 0.2 g/nm² or 0.2 units/nm² is administered to a tumor site. Tumor concentration of NTA at the tumor site may be at least 50%, 100%, 150%, 200%, 250%, 300%, 350%, 400%, 450% or 500% more than the tumor concentration of NTA with a PEG density of less than 0.2 g/nm² or 0.2 units/nm². In some embodiments, NTA comprising at least one PEG moiety and a PEG density of at least 0.5 g/nm² or 0.5 units/nm² is administered to a tumor site.

In some embodiments, the tumor site is selected from brain cancer, cervical cancer, esophageal cancer, gallbladder cancer, head and neck cancer, kidney cancer, liver cancer, multiple myeloma, thyroid cancer, lymphoma, renal cell carcinoma, leukemia, prostate cancer, lung cancer, pancreatic cancer, melanoma, colorectal cancer, ovarian cancer, breast cancer, glioblastoma multiforme and leptomeningeal carcinomatosis.

The present invention also provides a method of increasing the efficacy of NTA treatment comprising increasing tumor concentration of NTA by increasing PEG density of the nanoparticles.

The present invention also provides a population of nanoparticles having PEG density of between about 0.04 units/nm² or 0.04 g/nm² and about 3.0 units/nm² or 3.0 g/nm², inclusive. In some embodiments, the average diameter of the nanoparticles is between about 20 nm and about 999 nm, inclusive. In some embodiments, the nanoparticles comprise a therapeutic agent. In some embodiments, the nanoparticles comprise a polymer or lipid or a combination thereof. In some embodiments, the nanoparticles comprise a surfactant or lyoprotectant or a combination thereof. The term “population”, as used herein, is analogous to a plurality of members of that population.

The present invention also provides a method to predict tumor concentration of NTA comprising measuring tumor vasculature. The tumor concentration of NTA with a fixed PEG density depends on tumor vasculature. In some embodiments, tumor vasculature is measured with a fluorescently labeled pegylated macromolecule imaging agent such as AngioSense®. AngioSense® remains in the vasculature for extended periods of time and serves to provide details on the tumors that are investigated and how much the tumors are vascularized. In some embodiments, tumor vasculature has a positive correlation with tumor concentration of NTA. NTA tumor concentration is larger in tumors with a larger vasculature. In some embodiments, tumor vasculature has a negative correlation with tumor concentration of NTA. NTA tumor concentration is smaller in tumors with a larger vasculature.

### EXAMPLES

The following examples are intended to illustrate certain embodiments of the present teachings, but do not exemplify the full scope of the present teachings and therefore should not be construed to limit the scope of the present teachings.

### Example 1

### EPR Evaluation and Treatment of a Patient With Metastatic Lung Cancer

A patient with primary lung cancer that has progressed to metastatic stage will be indicated for treatment with compound 1 encapsulated in PLGA-PEG nanoparticles (1-NP).

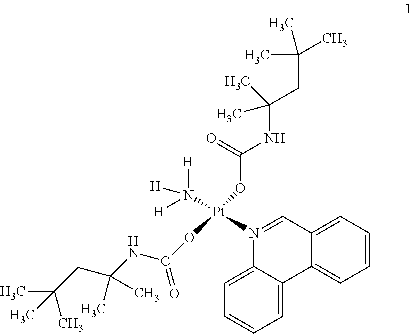

Imaging studies including PET scan will show two metastatic lesions around the dorsal root compressing the nerves. The compression of the nerves will cause a foot-drop syndrome in the patient.

To assess the EPR effect in the tumor sites, 102 mg of ferumoxytol is administered to the patient as a one time bolus. After an equilibration period of 90 minutes, the patient is imaged using Mill with both T₁ and T₂ imaging modalities. Assessment of the MRI image by a board-certified radiologist establishes that ferumoxytol had penetrated into all the tumor sites and the relative intensity of the ferumoxytol-associated image (ferumoxytol density) at the tumor site as compared to the surrounding tissue is greater than 10 to 1, with the metastatic tumors showing a higher relative intensity compared to surrounding tissue.

Based on the results from the imaging studies, the treating oncologist or other health care professional determines that tumors having elevated relative ferumoxytol density the assessment that all the tumor sites exhibited high EPR effect and the patient is a candidate for 1-NP treatment.

Subsequently, the patient is treated for six cycles of 1-NP treatment with each cycle consisting of one dose of 1 at 350 mg/m² on day 1 plus 1000 mg/m² of gemcitabine on days 1 and 8 of the cycle. The 1-NP particles have a drug loading of 5% and are engineered to release the drug at a medium release rate with most of the drug released from the nanoparticles within 72 hours. The nanoparticles are monodispersed with a mean diameter of 110 nm.

Follow up assessment of the patient demonstrates that all tumor sites demonstrating elevated relative ferumoxytol prior to treatment are responsive to the treatment using RECIST criteria with the metastatic sites. In some cases, tumors having the highest ferumoxytol relative density demonstrate the earliest detectable response to treatment, e.g., after two cycles of treatment. It is anticipated that by three weeks the foot drop in the patient starts to resolve and full functionality is be achieved by five weeks. By the end of the treatment at twelve weeks, the patient achieves a complete response.

This Example demonstrates a method of applying a method described herein.

### Example 2

### EPR Evaluation and Treatment of a Patient With Metastatic Colon Cancer

A patient with colon cancer that has previously been treated with one course of oxaliplatin is found to have progressive disease. While the primary tumor is stable, there are multiple metastases with two metastatic nodes, specifically, in the distal colon, that have aggressive growth rates. The treating oncologist is determining whether to initiate a new round of oxaliplain treatment in the form of a nanoparticle formulation of DACH-platin in PEG-PGLA nanoparticles (NPDP) or a whether to perform a radical colectomy. The NPDP is in the 40-50 nM size range and the DACH is conjugated to the PEG-PGLA.

To predict whether the patient will respond to the NPDP treatment, the oncologist refers the patient to a radiologist to assess the EPR effect in each of the tumor sites using ferumoxtran-10 (Combidex, Sinerem) iron oxide particles as a diagnostic agent. MRI assessment after ferumoxtran-10 infusion demonstrates high EPR effect in all the metastasis in the proximal colon but very low EPR in the distal colon, with the exception of one metastatic node in the distal tumor that exhibits medium level of EPR.

Based on the assessment of the EPR from the imaging study, the oncologist can reach a conclusion that it is unlikely that there will be enough accumulation of NPDP particles in the tumors, especially in the highly aggressive tumors in the distal colon, for effective treatment. Consequently, the oncologist makes a decision that the patient will not benefit sufficiently from the NPDP treatment and refers the patient to a surgeon for radical colonectomy. A timely decision will be critical due to the aggressive growth of the tumors.

### Example 3

### Synthesis of Polymeric Nanoparticles

Synthesis of Polylactide-Cyanine7 Conjugate Polymer

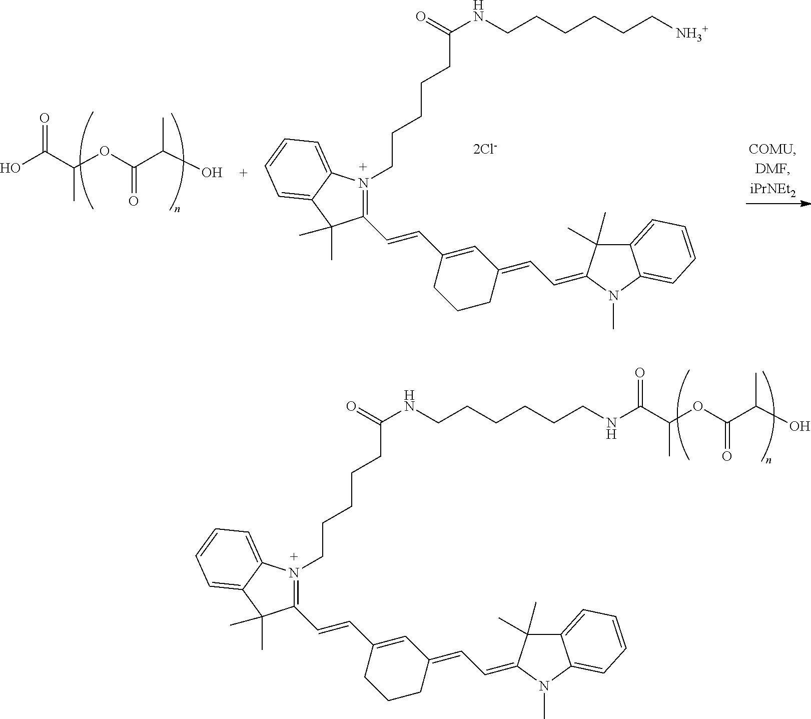

A flask was charged with polylactide polymer (PLA25) (525 mg, 0.0210 mmol), COMU (9.50 mg, 0.0232 mmol) and Cyanine7 amine (19.0 mg, 0.0232 mmol). DMF (5 mL) and diisopropylamine (0.10 mL) were added, and the reaction stirred in the dark at room temperature for 20 h. All solvent was removed in vacuo, and the remaining material dissolved in ethyl acetate (4 mL). This solution was added dropwise to a vial of 0° C. isopropanol (60 mL) with rapid stirring. The resulting suspension was centrifuged, and the supernatant decanted. This dissolve/precipitate/centrifuge sequence was repeated another two times, until very little green color was seen in the supernatant. After the final centrifugation, the remaining material was taken up in acetonitrile (5 mL), cooled to 0 oC, and water (2 mL) was added. The solution was quickly frozen, and lyophilized to give polylactide-Cy7 conjugate polymer (230 mg, 44% yield).

Formulation of Polylactide/Polylactide-Polyethylene glycol/Polylactide-Cy7 Nanoparticles With Low PEG Content and Small Particle Size (Polymeric Nanoparticle A)

Polylactide polymer (PLA25, Evonik, MW: 25 kDa, PDI: 1.8), polylactide polymer (PLA57, Evonik, MW: 57 kDa. PDI: 2.0), polylactide-block-methoxy-poly(ethylene glycol) (PLA69-mPEG5, Evonik, MW: 74 kDa, PDI: 1.7) and polylactide-Cy7 conjugate polymer at a weight ratio of 7.5/35/50/7.5 respectively were dissolved at a total polymer concentration of 80 mg/mL in ethyl acetate (Sigma Aldrich). The nanoparticles were formed using a single oil in water emulsion method. The polymer/copolymer/solvent solution was added to the aqueous phase (water containing 1.0% Tween saturated with ethyl acetate) at an organic to aqueous ratio of 1:10 and a coarse emulsion was prepared using an ultrasound bath and a rotor-stator homogenizer. The coarse emulsion was then processed through a high-pressure homogenizer (Microfluidics, operated at 10,000 psi) for N=4 passes to form a nanoemulsion. The nanoemulsion was quenched into a 20-fold dilution of cold water (0-5° C.) to remove a large portion of the ethyl acetate solvent resulting in hardening of the emulsion droplets and formation of a nanoparticle suspension. Tangential flow filtration (Spectrum, 500 kDa MWCO, mPES membrane) was used to concentrate and wash the nanoparticle suspension with water. A lyoprotectant, 10% sucrose (Sigma Aldrich), was added to the nanoparticle suspension. The formulation was stored frozen at ≤20° C. Particle size (Z-avg.) and the polydispersity indices (PDI) of the nanoparticles were characterized by dynamic light scattering, as summarized below in Table 1. UV-vis spectrophotometry was used at a wavelength of 760 nm to analyze the concentration of Cy7 in the nanoparticles and the PLA-mPEG content was determined by HPLC, both values are also summarized below in Table 1.

Formulation of Polylactide/Polylactide-Polyethylene glycol/Polylactide-Cy7 Nanoparticles With Low PEG Content and Large Particle Size (Polymeric Nanoparticle B)

Polylactide polymer (PLA25, Evonik, MW: 25 kDa, PDI: 1.8), polylactide polymer (PLA57, Evonik, MW: 57 kDa. PDI: 2.0), polylactide-block-methoxy-poly(ethylene glycol) (PLA69-mPEG5, Evonik, MW: 74 kDa, PDI: 1.7) and polylactide-Cy7 conjugate polymer at a weight ratio of 7.5/35/50/7.5 respectively were dissolved at a total polymer concentration of 50 mg/mL in ethyl acetate (Sigma Aldrich). The nanoparticles were formed using a single oil in water emulsion method. The polymer/copolymer/solvent solution was added to the aqueous phase (water containing 0.2% Tween saturated with ethyl acetate) at an organic to aqueous ratio of 1:10 and a coarse emulsion was prepared using an ultrasound bath and a rotor-stator homogenizer. The coarse emulsion was then processed through a high-pressure homogenizer (Microfluidics, operated at 10,000 psi) for N=4 passes to form a nanoemulsion. The nanoemulsion was quenched into a 20-fold dilution of cold water (0-5° C.) to remove a large portion of the ethyl acetate solvent resulting in hardening of the emulsion droplets and formation of a nanoparticle suspension. Tangential flow filtration (Spectrum, 500 kDa MWCO, mPES membrane) was used to concentrate and wash the nanoparticle suspension with water. A lyoprotectant, 10% sucrose (Sigma Aldrich), was added to the nanoparticle suspension. The formulation was stored frozen at ≤20° C. Particle size (Z-avg.) and the polydispersity index (PDI) of the nanoparticles were characterized by dynamic light scattering, as summarized below in Table 1. UV-vis spectrophotometry was used at a wavelength of 760 nm to analyze the concentration of Cy7 in the nanoparticles and the PLA-mPEG content was determined by HPLC, both values are also summarized below in Table 1.

Formulation of Polylactide/Polylactide-Polyethylene glycol/Polylactide-Cy7 Nanoparticles With High PEG Content and Small Particle Size (Polymeric Nanoparticle C)

Polylactide polymer (PLA25, Evonik, MW: 25kDa, PDI: 1.8), polylactide-block-methoxy-poly(ethylene glycol) (PLA11-mPEG5, Evonik, MW: 16 kDa, PDI: 1.1) and polylactide-Cy7 conjugate polymer at a weight ratio of 9.5/85/5.5 respectively were dissolved at a total polymer concentration of 80 mg/mL in ethyl acetate (Sigma Aldrich). The nanoparticles were formed using a single oil in water emulsion method. The polymer/copolymer/solvent solution was added to the aqueous phase (water containing 0.2% Tween saturated with ethyl acetate) at an organic to aqueous ratio of 1:10 and a coarse emulsion was prepared using an ultrasound bath and a rotor-stator homogenizer. The coarse emulsion was then processed through a high-pressure homogenizer (Microfluidics, operated at 10,000 psi) for N=4 passes to form a nanoemulsion. The nanoemulsion was quenched into a 20-fold dilution of cold water (0-5° C.) to remove a large portion of the ethyl acetate solvent resulting in hardening of the emulsion droplets and formation of a nanoparticle suspension. Tangential flow filtration (Spectrum, 500 kDa MWCO, mPES membrane) was used to concentrate and wash the nanoparticle suspension with water. A lyoprotectant, 10% sucrose (Sigma Aldrich) was added to the nanoparticle suspension. The formulation was stored frozen at ≤20° C. Particle size (Z-avg.) and the polydispersity index (PDI) of the nanoparticles were characterized by dynamic light scattering, as summarized below in Table 1. UV-vis spectrophotometry was used at a wavelength of 760 nm to analyze the concentration of Cy7 in the nanoparticles and the PLA-mPEG content was determined by HPLC, both values are also summarized below in Table 1.

Formulation of Polylactide/Polylactide-Polyethylene glycol/Polylactide-Cy7 Nanoparticles With High PEG Content and Small Particle Size (Polymeric Nanoparticle D)

Polylactide polymer (PLA25, Evonik, MW: 25 kDa, PDI: 1.8), polylactide-block-methoxy-poly(ethylene glycol) (PLA11-mPEG5, Evonik, MW: 16 kDa, PDI: 1.1) and polylactide-Cy7 conjugate polymer at a weight ratio of 7.5/88/4.5 respectively were dissolved at a total polymer concentration of 100 mg/mL in a solvent mixture of dichloromethane/ethyl acetate (Sigma Aldrich, 75%/25%). The nanoparticles were formed using a single oil in water emulsion method. The polymer/copolymer/solvent solution was added to the aqueous phase (water containing no emulsifier) at an organic to aqueous ratio of 1:10 and a coarse emulsion was prepared using an ultrasound bath and a rotor-stator homogenizer. The coarse emulsion was then processed through a high-pressure homogenizer (Microfluidics, operated at 10,000 psi) for N=4 passes to form a nanoemulsion. The nanoemulsion was quenched into a 20-fold dilution of cold water (0-5° C.) to remove a large portion of the ethyl acetate/dichloromethane solvent resulting in hardening of the emulsion droplets and formation of a nanoparticle suspension. Tangential flow filtration (Spectrum, 500 kDa MWCO, mPES membrane) was used to concentrate and wash the nanoparticle suspension with water. A lyoprotectant, 10% sucrose (Sigma Aldrich) was added to the nanoparticle suspension. The formulation was stored frozen at ≤20° C. Particle size (Z-avg.) and the polydispersity index (PDI) of the nanoparticles were characterized by dynamic light scattering, as summarized below in Table 1. UV-vis spectrophotometry was used at a wavelength of 760 nm to analyze the concentration of Cy7 in the nanoparticles and the PLA-mPEG content was determined by HPLC, both values are also summarized below in Table 1.

TABLE 1
Composition of Polymeric Nanoparticles

| | | | | Surface Charge | | |
|---|---|---|---|---|---|---|
| | Polymers Used | Particle Size | | (in water) | mPEG amount | PEG density |
| Nanoparticle | in Formulation | Z-avg | | Zeta potential | (Measured) | (Calculated) |
| # | (Initial composition) | (nm) | PDI | (mV) | (wt %) | (g/nm²) |
| A | PLA74-PEG5/PLA57/PLA25 | 46 | 0.1 | −21 | 2.8-4.0 | 0.04 |
| B | PLA74-PEG5/PLA57/PLA25 | 124 | 0.1 | −34 | 3.5 | 0.11 |
| C | PLA16-PEG5/PLA25 | 62 | 0.2 | −22 | 27.8 | 0.44 |
| D | PLA16-PEG5/PLA25 | 97 | 0.2 | −26 | 20.9 | 0.52 |

### Example 4

### Fluorescence Imaging of Fluorescently Labeled Polymeric Nanoparticles Co-localized with AngioSPARK

To assess the ability of AngioSPARK® 680 (PerkinElmer Inc., Boston, Mass.) to co-localize with Polymeric Nanoparticle D in an in vivo tumor xenograft model, we tested the effect of combined dosing of these two fluorescently labeled nanoparticles in a human A2780 ovarian xenograft and a human H460 NSCLC xenograft. In vivo xenograft imaging was performed over time using the FMT 2000 Fluorescence Tomography System (PerkinElmer Inc., Boston, Mass.). All mice were treated in accordance with the OLAW Public Health Service Policy on Human Care and Use of Laboratory Animals and the ILAR Guide for the Care and Use of Laboratory Animals, and studies were conducted at Blend Therapeutics (Watertown, Mass.). All in vivo studies were conducted following the protocols approved by the Blend Therapeutics Animal Care and Use Committee. All mice were fed Advanced Protocol® Verified 75 IF Irradiated (LabDiet, St. Louis, Mo.) mouse diet formulated with low soy isoflavone levels to minimize background fluorescence during in vivo imaging. For the A2780 in vivo studies, 10 week old female NCR nude mice were inoculated subcutaneously into the right flank with 1.0 million cells in 1:1 RPMI 1640 (Invitrogen, Carlsbad, Calif.)/Matrigel (BD Biosciences, San Jose, Calif.) For the H460 in vivo studies, 11 week old female NCR nude mice were inoculated subcutaneously into the right flank with 2.5 million cells in 1:1 RPMI 1640 (Life Technologies, Grand Island, N.Y., CA)/Matrigel (BD Biosciences, San Jose, Calif.). Tumor measurements were taken weekly, using vernier calipers. Tumor volume was calculated using the formula: V=½(width×width×length).

When tumors approached a volume of 500 mm³, mice were randomized into two groups of five animals. In the co-localization group, mice were treated with a combined dose solution of AngioSPARK® 680 and Polymeric Nanoparticle D at 4 nmol per mouse by intravenous injection. In a separate group, mice were dosed with AngioSense® 750 (PerkinElmer Inc., Boston, Mass.) at 2 nmol per mouse by intravenous injection. All mice were dosed one time only during the study. In vivo 3D (Isosurface, Volume Rendering and Slices) fluorescent images of the tumor xenograft were taken at 4 hours, 24 hours, 48 hours and 72 hours after dosing with AngioSPARK® 680 and Polymeric Nanoparticle D and each mouse scanned for both AngioSPARK® 680 on the 680 wavelength and for the Polymeric Nanoparticle D at the 750 wavelength. The AngioSense® 750 group was scanned for 3D tumor images at the 24 hour timepoint only at the 750 wavelength. A 3D scan was performed on a naive xenograft mouse on both the 680 and 750 wavelengths for use as a background control for tumor fluorescence. Ex vivo organ tissue was imaged after the 72 hour timepoint for both xenograft models.

Equivalent regions of interest (ROI) were measured in the tumor, liver, spleen and kidney for comparative fluorescence. Data analysis was completed on the FMT 2000 using TrueQuant (PerkinElmer Inc., Downers Grove, Ill.) imaging software. Five additional tumor xenografts were assessed using the FMT 2000 in vivo imaging with 3D scans taken at the 24 hour timepoint only. These xenografts were derived using either human NCI-H69 SCLC cells, human Calu-6 lung adenocarcinoma cells, human AsPC-1 pancreas adenocarcinoma cells or human NCI-H520 NSCLS cells. For the NCI-H69 in vivo studies, two xenograft studies were performed. In the first NCI-H69 tumor xenograft, 9 week old female NCR nude mice were inoculated subcutaneously into the right flank with 2.5 million cells in 1:1 RPMI 1640 (Life Technologies, Grand Island, N.Y.)/Matrigel (BD Biosciences, San Jose, Calif.). In the second NCI-H69 study 7 week old female NCR nude mice were inoculated subcutaneously into the right flank with 2.5 million cells in 1:1 RPMI 1640 (Life Technologies, Grand Island, N.Y.)/Matrigel (BD Biosciences, San Jose, Calif.). For the Calu-6 in vivo studies, 9 week old female NCR nude mice were inoculated subcutaneously into the right flank with 5.0 million cells in 1:1 MEM (Life Technologies, Grand Island, N.Y.)/Matrigel (BD Biosciences, San Jose, Calif.). For the AsPC-1 in vivo studies, 7 week old female NCR nude mice were inoculated subcutaneously into the right flank with 5.0 million cells in 1:1 RPMI 1640 (Life Technologies, Grand Island, N.Y.)/Matrigel (BD Biosciences, San Jose, Calif.). For the H520 in vivo studies, 8 week old female NCR nude mice were inoculated subcutaneously into the right flank with 5.0 million cells in 1:1 RPMI 1640 (Life Technologies, Grand Island, N.Y.)/Matrigel (BD Biosciences, San Jose, Calif.). Each of the single timepoint xenograft studies included a co-localization group in which mice were treated with a combined dose solution of AngioSPARK® 680 at 4 nmol per mouse by intravenous injection. They also included a separate group in which mice were dosed with AngioSense® 750 (PerkinElmer Inc., Boston, Mass.) at 2 nmol per mouse by intravenous injection.

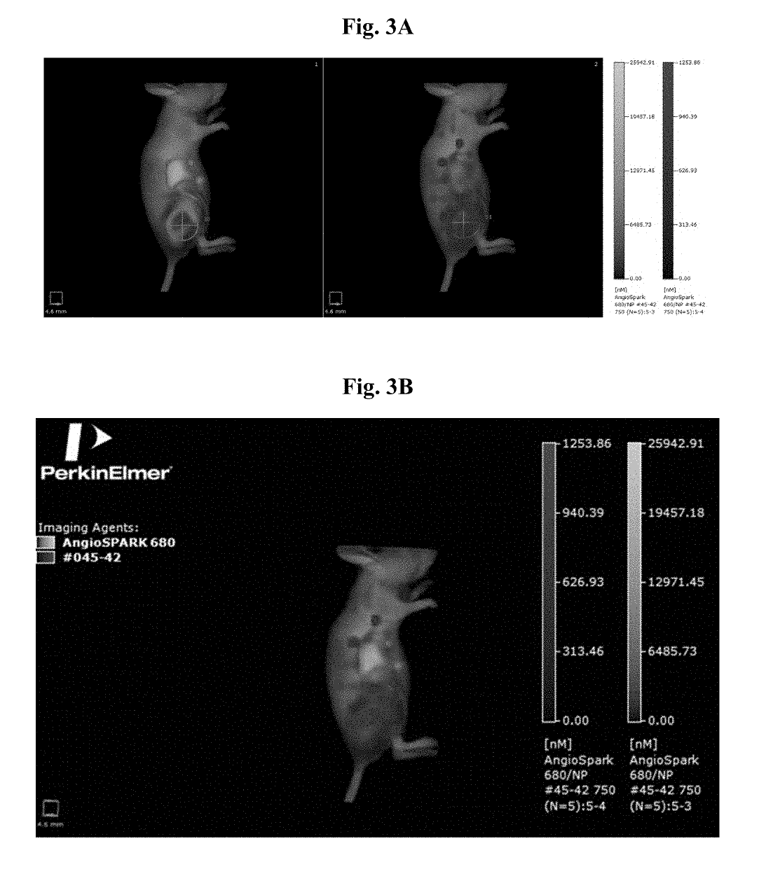

FIG. 3A shows same individual mouse (AN5) imaged on 680 and 750 wavelengths at 24 hours. FIG. 3B is a merged image of the images AngioSPARK and Polymeric Nanoparticle D in A2780 ovarian cancer xenogrqraphs at 72 hours. The total fluorescence in the region of interest and standard deviations are shown in Table 2 below (also see FIG. 3B). FIG. 3A and FIG. 3B show that AngioSPARK® and Polymeric Nanoparticle D co-localize in A2780 ovarian cancer xenografts in vivo.

TABLE 2
Total Fluorescence in Region of Interest and Standard
Deviations of AngioSPARK and Polymeric Nanoparticle D

| | Total Fluorescence | |
|---|---|---|
| | in Region of Interest | |
| Description | (pmol) | Std. dev. |
| AngioSPARK | 71.195 (n = 4) | 24.216 |
| Native control | 11.43 (n = 1) | NA |
| Polymeric Nanoparticle D | 99.03 (n = 5) | 28.377 |
| (NP #45-42) | | |
| Native control | 4.66 (n = 1) | NA |

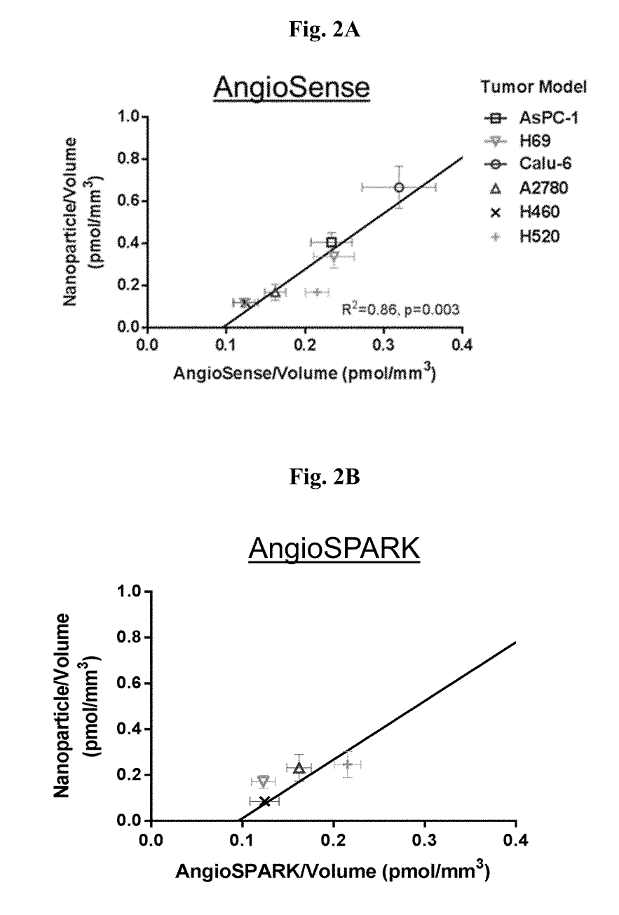

Total fluorescence of Polymeric Nanoparticle D, AngioSense, and AngioSPARK were measured in various tumor models including human pancreatic cancer (AsPC-1), human small cell lung carcinoma a small cell lung cancer (NCI-H69), human lung adenocarcinoma (Calu-6), human ovarian cancer (A2780), human lung cancer (H460), and human lung squamous cell carcinoma (NCI-H520) xenograft models. The results are shown in Table 3 (plotted in FIG. 2A and FIG. 2B). The level of accumulation of Polymeric Nanoparticle D has a positive correlation with the level of accumulation of both AngioSense and AngioSPARK. The results show a linear correlation between the level of accumulation of a contrast agent and the level of accumulation of nanoparticles, regardless of tumor type.

TABLE 3
Total Fluorescence in Region of Interest and Standard Deviations
of AngioSPARK, AngioSense\ and Polymeric Nanoparticle D

| | | | Std. Error |
|---|---|---|---|
| | | Mean (Fluorescence/ | (Fluorescence/ |
| | | tumor volume) | tumor volume) |
| Tumor type | Imaged Species | (pmol/mm³) | (pmol/mm') |
| A2780 | AngioSense | 0.162 | 0.014 |
| A2780 | AngioSPARK | 0.232 | 0.058 |
| A2780 | Polymeric NP D | 0.168 | 0.038 |
| AsPC-1 | AngioSense | 0.234 | 0.026 |
| AsPC-1 | AngioSPARK | TBD | TBD |
| AsPC-1 | Polymeric NP D | 0.406 | 0.047 |
| Calu-6 | AngioSense | 0.319 | 0.047 |
| Calu-6 | AngioSPARK | TBD | TBD |
| Calu-6 | Polymeric NP D | 0.667 | 0.100 |
| H460 | AngioSense | 0.124 | 0.016 |
| H460 | AngioSPARK | 0.086 | 0.008 |
| H460 | Polymeric NP D | 0.118 | 0.014 |
| H520 | AngioSense | 0.215 | 0.015 |
| H520 | AngioSPARK | 0.246 | 0.058 |
| H520 | Polymeric NP D | 0.168 | 0.011 |
| H69-1 | AngioSense | 0.237 | 0.026 |
| H69-1 | AngioSPARK | TBD | TBD |
| H69-1 | Polymeric NP D | 0.337 | 0.054 |
| H69-2 | AngioSense | 0.123 | 0.013 |
| H69-2 | AngioSPARK | 0.171 | 0.029 |
| H69-2 | Polymeric NP D | 0.116 | 0.021 |

Note:
Readings at 24 hour time point.

### Example 5

### Nanoparticle Tumor Concentration is Dependent on Nanoparticle PEG Density

A PEG assay was used to determine the amount of PEG present in polymeric nanoparticles labeled with a fluorescent dye. The PEG HPLC method was developed to determine the level of mPEG in the PEGylated polymeric nanoparticles. The method requires a hydrolysis step (digestion) of lyophilized nanoparticles followed by separation of mPEG from other components in the sample using HPLC linked to a charged aerosol detector 1N NaOH was used for the hydrolysis step, and was followed by neutralization of the NP solution using 1N HCl upon completion of digestion. The hydrolysis time had to be established for every PLA-PEG batch with different MW to assure that all mPEG molecules are being released from the PEG-PLA block polymer and that the mPEG fragment itself has not been degraded during the digestion. After digestion, sample was injected into an Agilent Zorbax Eclipse XDB-C18 3.5 micron particle size, 4.6×100 mm column for water/acetonitrile gradient separation. Charged aerosol detector was used for detecting mPEG moieties. Quantitation was achieved by comparison to a response factor derived from a calibration curve of an external PEG standard. Logarithmic transformations of the response and the concentration of the sample are used for calculation, as this weighing best model the response behavior of a CAD.

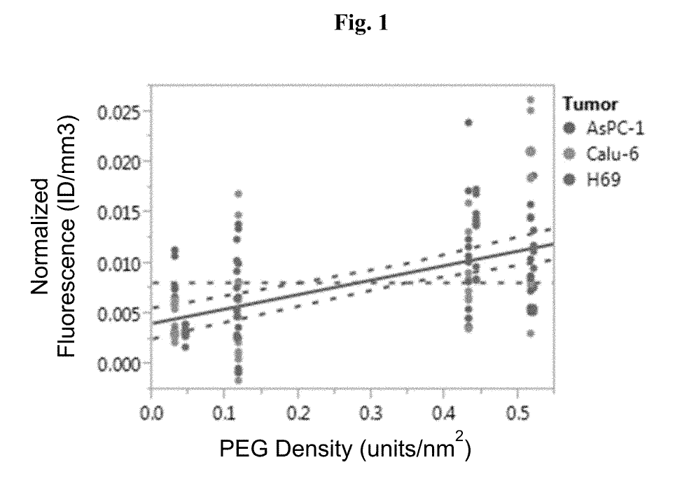

A series of calculations were then performed to determine the PEG density, namely from the particle size of the nanoparticles, the mass of each nanoparticle is determined. From this value, the amount of PEG per nanoparticle and the surface area per PEG were determined to calculate the PEG density. Fluorescence of labeled polymeric nanoparticles was measured in various tumor models, including AsPC-1, Calu-6, and H69. Nanoparticle tumor concentration in different tumors (H69-1, Calu-6 and AsPC-1) and nanoparticle PEG density are shown in Table 4 below and plotted in FIG. 1. FIG. 1 shows that nanoparticle PEG density has a statistically significant effect on tumor concentration (p<0.001). Tumor concentration of nanoparticles increases with particle size in highly vascularized tumors (p=0.05).

TABLE 4
Tumor volume, normalized fluorescence/tumor
volume, and PEG density

| | Tumor | Normalized Fluorescence/ | |
|---|---|---|---|
| | volume | Tumor Volume | PEG density |
| Tumor type | (mm³) | (Initial dose/mm³) | (units/nm²) |
| H69-1 | 199.01 | 0.0020 | 0.04 |
| H69-1 | 244.97 | 0.0028 | 0.04 |
| H69-1 | 254.85 | 0.0024 | 0.04 |
| H69-1 | 293.21 | 0.0022 | 0.04 |
| H69-1 | 293.41 | 0.0023 | 0.04 |
| H69-1 | 201.77 | 0.0089 | 0.11 |
| H69-1 | 240.05 | 0.0110 | 0.11 |
| H69-1 | 261.54 | 0.0062 | 0.11 |
| H69-1 | 292.92 | 0.0078 | 0.11 |
| H69-1 | 294.68 | 0.0051 | 0.11 |
| H69-1 | 206.53 | 0.0137 | 0.44 |
| H69-1 | 237.58 | 0.0133 | 0.44 |
| H69-1 | 262.62 | 0.0129 | 0.44 |
| H69-1 | 286.96 | 0.0076 | 0.44 |
| H69-1 | 295.25 | 0.0166 | 0.44 |
| H69-1 | 215.43 | 0.0101 | 0.52 |
| H69-1 | 237.58 | 0.0040 | 0.52 |
| H69-1 | 267.89 | 0.0044 | 0.52 |
| H69-1 | 284.65 | 0.0043 | 0.52 |
| H69-1 | 298.42 | 0.0106 | 0.52 |
| Calu-6 | 194.08 | 0.0009 | 0.04 |
| Calu-6 | 225.25 | 0.0048 | 0.04 |
| Calu-6 | 225.66 | 0.0043 | 0.04 |
| Calu-6 | 272.03 | 0.0017 | 0.04 |
| Calu-6 | 275.68 | 0.0020 | 0.04 |
| Calu-6 | 199.89 | 0.0048 | 0.11 |
| Calu-6 | 224.05 | 0.0174 | 0.11 |
| Calu-6 | 229.99 | 0.0194 | 0.11 |
| Calu-6 | 267.19 | 0.0089 | 0.11 |
| Calu-6 | 279.91 | 0.0107 | 0.11 |
| Calu-6 | 200.82 | 0.0151 | 0.44 |
| Calu-6 | 218.18 | 0.0124 | 0.44 |
| Calu-6 | 241.60 | 0.0077 | 0.44 |
| Calu-6 | 261.36 | 0.0055 | 0.44 |
| Calu-6 | 285.89 | 0.0027 | 0.44 |
| Calu-6 | 206.33 | 0.0274 | 0.52 |
| Calu-6 | 208.25 | 0.0068 | 0.52 |
| Calu-6 | 247.93 | 0.0263 | 0.52 |
| Calu-6 | 257.78 | 0.0157 | 0.52 |
| Calu-6 | 291.44 | 0.0195 | 0.52 |
| H69-1 | 403.03 | 0.0032 | 0.04 |
| H69-1 | 896.98 | 0.0026 | 0.04 |
| H69-1 | 1267.75 | 0.0010 | 0.04 |
| H69-1 | 799.08 | 0.0025 | 0.04 |
| H69-1 | 970.62 | 0.0033 | 0.04 |
| H69-1 | 333.61 | 0.0039 | 0.11 |
| H69-1 | 573.46 | 0.0041 | 0.11 |
| H69-1 | 1027.08 | 0.0012 | 0.11 |
| H69-1 | 1282.61 | 0.0016 | 0.11 |
| H69-1 | 949.62 | 0.0034 | 0.11 |
| H69-1 | 318.87 | 0.0089 | 0.44 |
| H69-1 | 694.13 | 0.0141 | 0.44 |
| H69-1 | 415.42 | 0.0161 | 0.44 |
| H69-1 | 964.36 | 0.0075 | 0.44 |
| H69-1 | 587.07 | 0.0130 | 0.44 |
| H69-1 | 521.04 | 0.0175 | 0.52 |
| H69-1 | 819.80 | 0.0066 | 0.52 |
| H69-1 | 967.55 | 0.0064 | 0.52 |
| H69-1 | 950.47 | 0.0084 | 0.52 |
| H69-1 | 499.57 | 0.0122 | 0.52 |
| Calu-6 | 470.50 | 0.0051 | 0.04 |
| Calu-6 | 481.16 | 0.0026 | 0.04 |
| Calu-6 | 1499.62 | 0.0019 | 0.04 |
| Calu-6 | 1358.36 | 0.0015 | 0.04 |
| Calu-6 | 623.18 | 0.0050 | 0.04 |
| Calu-6 | 932.78 | 0.0074 | 0.11 |
| Calu-6 | 1621.80 | 0.0037 | 0.11 |
| Calu-6 | 1578.80 | 0.0040 | 0.11 |
| Calu-6 | 1735.17 | 0.0032 | 0.11 |
| Calu-6 | 2123.27 | 0.0010 | 0.11 |
| Calu-6 | 1160.61 | 0.0065 | 0.44 |
| Calu-6 | 1002.76 | 0.0059 | 0.44 |
| Calu-6 | 915.75 | 0.0038 | 0.44 |
| Calu-6 | 1439.52 | 0.0031 | 0.44 |
| Calu-6 | 773.11 | 0.0083 | 0.44 |
| Calu-6 | 392.98 | 0.0196 | 0.52 |
| Calu-6 | 657.24 | 0.0157 | 0.52 |
| Calu-6 | 744.06 | 0.0223 | 0.52 |
| Calu-6 | 1194.89 | 0.0042 | 0.52 |
| Calu-6 | 1039.02 | 0.0091 | 0.52 |
| AsPC-1 | 178.69 | 0.0065 | 0.04 |
| AsPC-1 | 208.32 | 0.0062 | 0.04 |
| AsPC-1 | 209.61 | 0.0094 | 0.04 |
| AsPC-1 | 182.48 | 0.0161 | 0.11 |
| AsPC-1 | 197.80 | 0.0023 | 0.11 |
| AsPC-1 | 219.42 | 0.0126 | 0.11 |
| AsPC-1 | 270.33 | 0.0108 | 0.11 |
| AsPC-1 | 291.96 | 0.0092 | 0.11 |
| AsPC-1 | 185.18 | 0.0231 | 0.44 |
| AsPC-1 | 197.50 | 0.0115 | 0.44 |
| AsPC-1 | 225.25 | 0.0109 | 0.44 |
| AsPC-1 | 257.49 | 0.0164 | 0.44 |
| AsPC-1 | 314.45 | 0.0094 | 0.44 |
| AsPC-1 | 189.97 | 0.0170 | 0.52 |
| AsPC-1 | 195.38 | 0.0116 | 0.52 |
| AsPC-1 | 226.73 | 0.0084 | 0.52 |
| AsPC-1 | 251.57 | 0.0156 | 0.52 |
| AsPC-1 | 316.20 | 0.0097 | 0.52 |
| AsPC-1 | 327.13 | 0.0021 | 0.04 |
| AsPC-1 | 473.18 | 0.0017 | 0.04 |
| AsPC-1 | 447.85 | 0.0102 | 0.04 |
| AsPC-1 | 782.66 | 0.0021 | 0.04 |
| AsPC-1 | 307.04 | 0.0054 | 0.04 |
| AsPC-1 | 291.52 | 0.0165 | 0.11 |
| AsPC-1 | 271.72 | 0.0019 | 0.11 |
| AsPC-1 | 413.40 | 0.0053 | 0.11 |
| AsPC-1 | 573.01 | 0.0017 | 0.11 |
| AsPC-1 | 374.72 | 0.0063 | 0.11 |
| AsPC-1 | 495.34 | 0.0074 | 0.44 |
| AsPC-1 | 387.02 | 0.0100 | 0.44 |
| AsPC-1 | 382.87 | 0.0046 | 0.44 |
| AsPC-1 | 566.27 | 0.0037 | 0.44 |
| AsPC-1 | 386.37 | 0.0028 | 0.44 |
| AsPC-1 | 562.77 | 0.0066 | 0.52 |
| AsPC-1 | 436.14 | 0.0094 | 0.52 |
| AsPC-1 | 400.32 | 0.0067 | 0.52 |
| AsPC-1 | 467.60 | 0.0099 | 0.52 |
| AsPC-1 | 499.79 | 0.0064 | 0.52 |

TABLE 5
Summary of Fit for FIG. 1

| | |
|---|---|
| RSquare | 0.314402 |
| RSquare Adj | 0.290133 |
| Root Mean Square Error | 0.004917 |
| Mean of Response | 0.008146 |
| Observations (or Sum Wgts) | 118 |

TABLE 6
Parameters Estimates of FIG. 1

| | | | t | Prob > |
|---|---|---|---|---|
| Term | Estimate | Std Error | Ratio | [t] |
| Intercept | 0.0018029 | 0.00165 | 1.09 | 0.2768 |
| Tumor(H69-AsPC-1&Calu-6) | −0.000714 | 0.000478 | −1.49 | 0.1385 |
| PS (nm) | 0.0000245 | 0.000017 | 1.44 | 0.1533 |
| PEG (g/nm\hat{ }2) | 0.01432 | 0.002221 | 6.45 | <.0001* |
| Tumor(H69-AsPC-1&Calu- | −3.387e−5 | 0.000017 | −1.99 | 0.0494* |
| 6)*(PS(nm)-84.7627) | | | | |

### Example 6

### Nanoparticle Tumor Concentration is Dependent on Vasculature

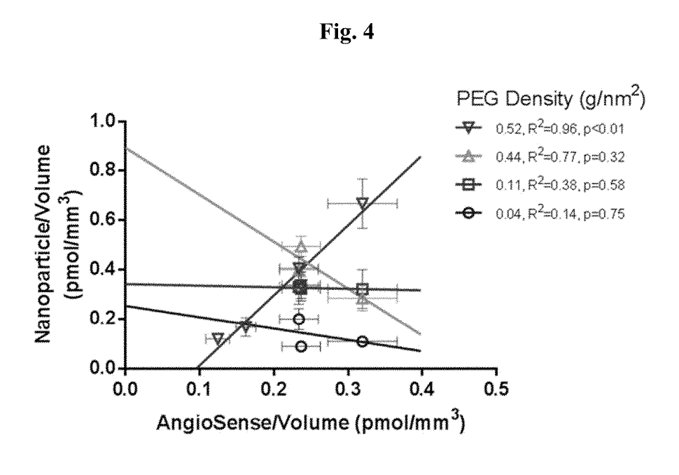

Tumor vasculature was characterized by fluorescently labeled PEGylated macromolecule AngioSense and plotted against tumor concentration of nanoparticles with different PEG densities. Results are shown in FIG. 4. For Polymeric Nanoparticle D (PEG density=0.52), there is a positive and statistically significant correlation between nanoparticle concentration and AngioSense concentration (R²=0.96, p<0.01).

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein are representative of the subject matter which is broadly contemplated by the present invention. It is further understood that the scope of the present invention is not intended to be limited to the embodiment shown herein but is to be accorded the widest scope consistent with the patent law and the principles and novel features disclosed herein.

All references, including publications, patent applications, and patents, cited herein are hereby incorporated by reference to the same extent as if each reference were individually and specifically indicated to be incorporated by reference and were set forth in its entirety herein.

Alternative embodiments of the claimed disclosure are described herein. Of these, variations of the disclosed embodiments will become apparent to those of ordinary skill in the art upon reading the foregoing disclosure. The inventors expect skilled artisans to employ such variations as appropriate (e.g., altering or combining features or embodiments), and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

We claim:

1. A method of selecting a subject to be treated with a nanoparticle therapeutic agent (NTA), the method comprising:
(a) administering a contrast agent to the subject;
(b) measuring the level of accumulation of the contrast agent at at least one intended site of treatment; and
(c) selecting the subject for NTA treatment based on the level of the accumulation of the contrast agent;
wherein the intended site of treatment is a tumor.

2. The method of claim 1, wherein the contrast agent and NTA differ from one another based on at least one selected parameter by at least 2 fold.

3. The method of claim 2, wherein the selected parameters are size, density, or surface charge.

4. The method of claim 1, further comprising comparing the level of accumulation at the intended site of treatment to a reference, and treating the subject with the NTA if there is an increase in the level of accumulation compared to the reference.

5. The method of claim 4, wherein the reference is measured in the subject at a reference site and the reference site is plasma, bone, or muscle.

6. The method of claim 1, wherein the contrast agent comprises a moiety selected from a fluorescent moiety, a luminescent moiety, a radioactive moiety, and a magnetic moiety.

7. The method of claim 6, wherein the contrast agent is ferumoxytol, AngioSense®, or AngioSPARK®.

8. The method of claim 1, wherein the level of accumulation of the contrast agent is measured with an imaging technique selected from ultrasound, X-ray, single-photon emission tomography/computed tomography (SPECT/CT), positron emission tomography/computed tomography (PET/CT), positron emission tomography (PET), magnetic resonance imaging (MRI), computed tomography (CT), single-photon emission tomography (SPECT), fluorescence tomography, and fluorescence spectroscopy.

9. The method of claim 1, wherein the tumor is pancreatic cancer, lung cancer, or ovarian cancer.

10. The method of claim 1, wherein the NTA comprises a conjugate having the formula:
(X—Y—Z)
wherein:
X is a targeting ligand;
Y is a linker; and
Z is a pharmaceutically active agent.

11. A method of treating cancer comprising:
(a) administering a contrast agent to a subject;
(b) measuring the level of accumulation of the contrast agent at an intended site of treatment, wherein the intended site of treatment is a tumor site;
(c) determining if the subject is suitable for NTA treatment based on the measured level of accumulation of the contrast agent; and
(d) administering NTA to the subject if the subject is determined to be suitable for NTA treatment based on the measured level of accumulation of the contrast agent.

12. The method of claim 11, wherein in the measured level of accumulation of the contrast agent is compared with a predetermined level.

13. The method of claim 11, further comprising measuring the level of accumulation of the contrast agent at a reference site and comparing the level of accumulation of the contrast agent at the intended site of treatment to the level of accumulation of contrast agent at the reference site.

14. The method of claim 13, wherein the reference site is plasma, bone, or muscle.

15. A method of assessing the efficacy of an NTA in treating a subject with cancer comprising:
(a) administering a contrast agent to a subject before treatment with an NTA;
(b) performing a pre-treatment imaging evaluation of the contrast agent in at least one intended site of treatment;
(c) administering the NTA to the subject;
(d) administering the contrast agent to the subject after NTA treatment;
(e) performing a post-treatment imaging evaluation of the intended site of treatment; and
(f) identifying any change in the post-treatment imaging evaluation compared to the pre-treatment imaging evaluation, wherein a decrease in the amount of contrast agent post-treatment in the targeted region indicates the NTA is an effective treatment, and
wherein the intended site of treatment is a tumor site.

16. The method of claim 15, wherein the imaging evaluation is performed with a diagnostic device selected from an ultrasound, fluorescence spectrometer, X-ray, MRI scanner, PET scanner, fluorescence tomography scanner, or CT scanner.

17. The method of claim 15, wherein the contrast agent is ferumoxytol, AngioSense®, or AngioSPARK®.

18. A method of increasing the accumulation of a nanoparticle at a tumor site, the method comprising administering a nanoparticle to the tumor site, wherein the nanoparticle comprises at least one PEG moiety and a PEG density of at least 0.2 g/nm² or 0.2 units/nm², wherein the accumulation is increased compared to accumulation of a nanoparticle that has a density of less than 0.2 g/nm² or 0.2 units/nm².

19. The method of claim 18, wherein the nanoparticle is a nanoparticle therapeutic agent (NTA) and comprises at least one pharmaceutically active agent.

20. The method of claim 18, wherein the PEG density of the nanoparticle is at least 0.5 g/nm² or 0.5 units/nm².

21. The method of claim 18, wherein the tumor is a highly vascularized tumor.

22. The method of claim 21, wherein the tumor site is ovarian, pancreatic or lung.
